(19) United States
(12) Patent Application Publication
(10) Pub. No.: US 2008/0249124 A1
(43) Pub. Date: Oct. 9, 2008

(54) WORTMANNIN-RAPALOG CONJUGATE AND USES THEREOF

(75) Inventors: Jianxin GU, River Edge, NJ (US); Ker Yu, Pine Brook, NJ (US); Judy Lucas, Nanuet, NY (US); Mark Ruppen, Garnerville, NY (US); Arie Zask, New York, NY (US); Semiramis Ayral-Kaloustian, Tarrytown, NY (US)

Correspondence Address: HOWSON AND HOWSON/WYETH;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 12/061,976

(22) Filed: Apr. 3, 2008

Related U.S. Application Data
(60) Provisional application No. 60/921,909, filed on Apr. 5, 2007.

(51) Int. Cl. A61K 31/436; A61K 31/407; C07D 267/00; A61P 35/04

(52) U.S. Cl. 514/291; 514/411; 540/468

(57) ABSTRACT

An anti-neoplastic wortmannin conjugate is described formed by a linking a rapalog and wortmannin. Such a linkage is removable following administration to a subject. Use of such a conjugate in an antineoplastic regimen is described.

### CROSS-REFERENCE TO RELATED APPLICATIONS

This application claims the benefit of the priority of U.S. Provisional Patent Application No. 60/921,909, filed Apr. 5, 2007.

### BACKGROUND OF THE INVENTION

The present invention relates to rapalog-wortmannin conjugates having anti-tumor activity.

Wortmannins and Rapamycins are two classes of highly potent and specific inhibitors of phosphatidylinositol-3(OH)-kinase (PI3K) and mTOR, respectively. PI3K is a heterodimeric enzyme comprised of the p85 regulatory and p110 catalytic subunits. In response to growth factor receptor stimulation, PI3K catalyzes the production of the lipid second messenger phosphatidylinositol-3,4,5-triphosphate (PIP3) at the cell membrane. PIP3 in turn contributes to the activation of a wide range of downstream cellular substrates. The most critical signaling mediators downstream of PI3K include the serine/threonine kinase AKT and the mammalian target of rapamycin (mTOR). AKT confers a dominant survival signal and promotes proliferation via direct phosphorylation of multiple cell death/apoptosis proteins and cell cycle factors. mTOR is a central regulator of cell growth via controlling cellular protein translation. Thus, the PI3K/AKT/TOR pathway is critical for cell proliferation, growth, survival and angiogenesis.

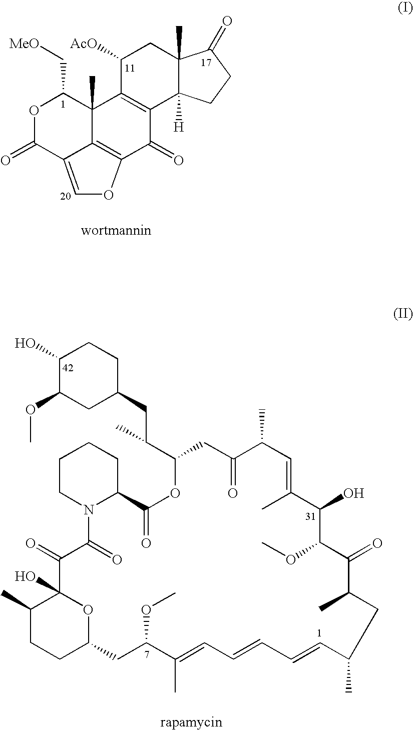

Wortmannin (formula (I)) is an irreversible inhibitor, at nanomolar concentrations, of PI3K that binds to a lysine in the ATP binding pocket of PI3K via opening of the electrophilic furan ring at its C-20 position and has been reported to have antitumor activity against tumor xenografts in animals. [Schultz, R. M., et al., (1995) “In vitro and in vivo antitumor activity of the phosphatidylinositol-3-kinase inhibitor, wortmannin” Anticancer Res., 15, 1135-1140]. The syntheses and SAR studies of wortmannin derivatives in efforts to search for analogs with improved properties over wortmannin have been described previously. For example, a 17β-Hydroxywortmannin prepared from the reduction of wortmannin with diborane, showed a 10-fold increase in activity relative to wortmannin and pushed the PI3K IC₅₀ into the subnanomolar range, with an IC₅₀ of 0.50 nM. However, anti-tumor activity of 17β-Hydroxywortmannin in the C3H mammary model showed no inhibition at a dose of 0.5 mg/kg and toxicity at a dose of 1.0 mg/kg. These findings led the authors to conclude that nucleophilic addition to the electrophilic C-20 position of wortmannin and related analogs is required for inhibitor potency and antitumor activity, but that this mechanism appears linked to the observed toxicity. [Norman, Bryan H., et al., (1996) “Studies on the Mechanism of Phosphatidylinositol 3-Kinase Inhibition by Wortmannin and Related Analogs,” J. Med. Chem., 39, 1106-1111, 1109-1110].

Recently, it was found that pegylated 17β-hydroxywortmannin (PEG-17-HWT conjugate) demonstrated an increased tolerability as compared to 17β-hydroxywortmannin in vivo, [Yu K, et al., (2005), “PWT-458, A Novel Pegylated-17-Hydroxywortmannin, Inhibits Phosphatidylinositol 3-Kinase Signaling and Suppresses Growth of Solid Tumors” Cancer Biol Ther. 4(5)].

Acetylation of 17β-hydroxywortmannin at its C-17 hydroxyl site showed a dramatic loss in activity leading the authors to conclude, “the active site cannot accommodate liphophilicity or steric bulk at C-17.” [Creemer, L. C., et al., (1996) “Synthesis and in Vitro Evaluation of New Wortmannin Esters: Potent Inhibitors of Phosphatidylinositol 3-Kinase,” J. Med. Chem., 39, 5021-5024, 5022]. This conclusion is consistent with the X-ray crystallographic structure of PI3K bound to wortmannin subsequently elucidated [Walker, Edward H., et. al. (2000) “Structural Determinants of Phosphoinositide 3-Kinase Inhibition by Wortmannin, LY294002, Quercetin, Myricetin, and Staurosporine,” Molecular Cell, 6(4), 909-919].

Other wortmannin derivatives are C-20 ring-opened compounds. By reacting wortmannin with nucleophiles at the C-20 position, the furan ring is opened. Such ring-opened compounds demonstrate a range of biological activities with improved toxicity and biological stability [Wipf, Peter, et al., (2004) “Synthesis and biological evaluation of synthetic viridins derived from C(20)-heteroalkylation of the steroidal PI-3-kinase inhibitor wortmannin,” Org. Biomol. Chem., 2, 1911-1920]. See also US Published Patent Application No. US2003/0109572 to Powis.

Rapamycin (sirolimus—formula (II)) is a lipophilic macrolide produced by Streptomyces hygroscopicus NRRL 5491 [U.S. Pat. Nos. 3,929,992; 3,993,749] with a 1,2,3-tricarbonyl moiety linked to a pipecolic acid lactone. Other related macrolides include FK506 (tacrolimus), FK520 (ascomycin or immunomycin), FK525, FK523, antascomicins, and meridamycin.

Rapamycin is a potent immunosuppressant with established or predicted therapeutic applications in the prevention of organ allograft rejection and in the treatment of autoimmune diseases. Rapamycin and related compounds for example, but without limitation, FK506, FK520, FK523, meridamycin, antascomicin, FK525 and tsukubamycin can be considered “FKBP-ligands”. The pharmacologic actions of rapamycin characterised to date are believed to be mediated by the interaction with cytosolic receptors termed FKBPs or immunophilins (this term is used to denote immunosuppressant binding proteins).

A range of synthesised rapamycin analogues using the chemically available sites of the molecule has been reported. Chemically available sites on the molecule for derivatisation or replacement include C40 and C₂₈ hydroxyl groups (e.g. U.S. Pat. Nos. 5,665,772 and 5,362,718), C39 and C16 methoxy groups (e.g. International Patent Publication No. WO 96/141807; U.S. Pat. No. 5,728,710), C32, C26 and C9 keto groups (e.g. U.S. Pat. Nos. 5,378,836; 5,138,051; 5,665,772). Hydrogenation at C17, C19 and/or C21, targeting the triene, resulted in retention of antifungal activity but loss of immunosuppression (e.g., U.S. Pat. Nos. 5,391,730 and 5,023,262). Significant improvements in the stability of the molecule (e.g. formation of oximes at C32, C40 and/or C28, U.S. Pat. Nos. 5,563,145; 5,446,048), resistance to metabolic attack (e.g., U.S. Pat. No. 5,912,253), bioavailability (e.g., U.S. Pat. Nos. 5,221,670; 5,955,457; International Patent Publication No. WO98/04279) and the production of prodrugs (e.g., U.S. Pat. Nos. 6,015,815; 5,432,183) have been achieved through derivatisation.

Rapamycin analogues have been prepared using recombinant strains that contain biosynthetic clusters, where one or more rapamycin polyketide synthase gene has been deleted or deactivated (e.g., International Patent Publication Nos. WO 2004/007709; WO2006/016167). These novel rapamycin analogues are referred to as “rapalogs”. Preclinical studies of rapamycin and rapalogs thereof have determined potency against many solid tumor types including breast, colon, prostate and renal cell carcinomas.

What are needed are alternative therapies for treatment of neoplasms.

### SUMMARY OF THE INVENTION

The invention provides a rapalog-wortmannin conjugate. In one embodiment, the conjugate is characterized by having the formula:

Rapalog-L-Wort

or a pharmaceutically acceptable salt or hydrate thereof, wherein Rapalog is defined herein; Wort is a wortmannin, and L is a linker which is bound to the rapalog and the wortmannin.

In one embodiment, the rapalog is a 41-Desmethoxyrapamycin.

In a further embodiment, the conjugate has the structure:

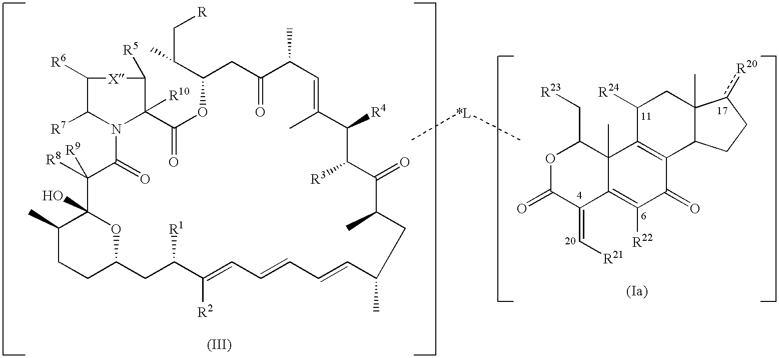

where the R groups and linker are as defined below. Suitably, following administration of a conjugate to a subject, the linker is partially, or completely, removed from one or both of the rapalog or the wortmannin.

In another aspect, the invention provides a composition comprising the rapalog-wortmannin conjugate and a pharmaceutically acceptable carrier.

In still another aspect, the invention provides for the use of a rapalog-wortmannin conjugate for the preparation of a medicament useful in antineoplastic therapy.

Still other aspects and advantages of the invention will be readily apparent to one of skill in the art.

### BRIEF DESCRIPTION OF THE FIGURES

FIG. 1 provides a line graph illustrating the in vivo efficacy of N,N,N′-trimethyl-1,3-propanediamine adduct of 42,17′-linked 41-Desmethoxyrapamycin-suberate-wortmannin conjugate (conjugate of Example 2b; ▴). D5W vehicle (glucose-water) served as the control ().

FIG. 2 provides a line graph illustrating the in vivo efficacy of the diallylamine adduct of 42,17′-linked 41-Desmethoxyrapamycin-suberate-wortmannin conjugate (conjugate of Example 2a; (▾); dashed line) as compared with 41-Desmethoxyrapamycin (▴; solid line), diallylamine adduct of 17-hydroxywortmannin (▪; solid line), and a mixture (♦; solid line) of 41-Desmethoxyrapamycin/diallylamine adduct of 17-hydroxywortmannin. D5W vehicle (glucose-water) served as the control (; solid line).

### DETAILED DESCRIPTION OF THE INVENTION

In one embodiment, the invention provides a conjugate formed by linking a rapalog and a wortmannin via a linker. Suitably, following administration of a conjugate to a subject, the linker is removed in whole or part from one or both of the rapalog or the wortmannin.

The Rapalog-L-Wort conjugates of the invention are anticipated to provide distinct advantages over either single agent or the physical (non-linked) combination of the two. Without wishing to be bound by theory, it is believed that the covalent linking of a wortmannin to a rapalog will improve the solubility over the rapalog alone. Improved solubility has important implications in clinical development and formulation. Further, as cancer therapies will likely consist of a cocktail of various combinations of compositions and standard chemotherapies, the simplicity of using a single, water-soluble dual inhibitor, rather than two separate inhibitors that require two different clinical formulations, is advantageous from perspective of formulating the compounds. Moreover, the conjugates are anticipated to outperform either single agents and also outperform physical combination of the two.

As defined herein, the term “a rapalog” defines a class of rapamycin analogues which are immunosuppressive compounds containing the rapalog nucleus of formula (III):

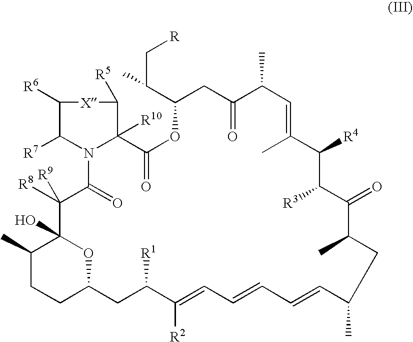

wherein, R¹ is selected from among OH, an ester, and an ether; R² is methyl or H; R³ is selected from among H, OH, an ester, and an ether; R⁴ is selected from among OH, an ester, an ether, an amide, a carbonate, a carbamate, and a phosphate; R⁵, R⁶, and R⁷ are independently selected from among H, alkyl, halo, and hydroxyl; R⁸, R⁹ is H, H or is O; R¹⁰ is selected from among H, alkyl, halo and hydroxyl; X″ is a bond or CHR¹¹; or —CHR⁵—X″—CHR⁶— is

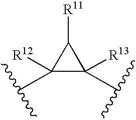

R¹¹, R¹², and R¹³ are independently selected from among H, alkyl, halo, and hydroxyl; R is selected from among:

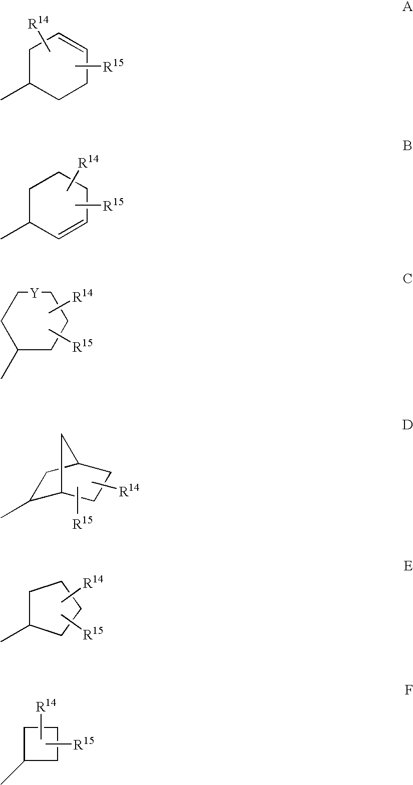

R¹⁴ and R¹⁵ are independently selected from among H, OH, halogen, thiol, amine, alkyl, an ester, an ether, an amide, a carbonate, a carbamate, a sulfonate, a phosphate, and a tetrazole; Y is a bond or CHR¹⁶; R¹⁶ is selected from among H, alkyl, halogen, and hydroxyl; with the proviso that the compounds do not include the following:

i) where R¹ is OCH₃ in combination with R² is CH₃, R³ is OCH₃, R⁴ is OH, R⁵ is H, R⁶ is H, R⁷ is H, R⁸, R⁹ is keto (═O), R¹⁰ is H, X is CH₂, R is formula C, Y is a bond, R¹⁴ is cis-3-OMe, R¹⁵ is trans-4-OH (rapamycin)

ii) where R¹ is OCH₃ in combination with R² is CH₃, R³ is OCH₃, R⁴ is OH, R⁵ is H, R⁶ is H, R⁷ is H, R⁸, R⁹ is keto, R¹⁰ is H, X is CH₂, R is formula C, Y is a bond, R¹⁴ is cis-3-OMe, R¹⁵ is a trans-4-ether such as trans-4-O—CH₂CH₂—OH (RAD001)

iii) where R¹ is OCH₃ in combination with R² is CH₃, R³ is OCH₃, R⁴ is OH, R⁵ is H, R⁶ is H, R⁷ is H, R⁸, R⁹ is keto, R¹⁰ is H, X is CH₂, R is formula C, Y is a bond, R¹⁴ is cis-3-OMe, R¹⁵ is a trans-4-ester such as trans-4-O—COC(CH₃)(CH₂OH)₂ (CCI779)

iv) where R¹ is OCH₃ in combination with R² is CH₃, R³ is OCH₃, R⁴ is OH, R⁵ is H, R⁶ is H, R⁷ is H, R⁸, R⁹ is keto, R¹⁰ is H, X is CH₂, R is formula C, Y is a bond, R¹⁴ is cis-3-OMe, R¹⁵ is cis-4-tetrazole (ABT578).

In another embodiment, this formula may exclude compounds

v) where R¹ is OCH₃ in combination with R² is CH₃, R³ is OCH₃, R⁴ is OH, R⁵ is H, R⁶ is H, R⁷ is H, R⁸, R⁹ is keto, R¹⁰ is H, X is CH₂, R is formula C, Y is a bond, R¹⁴ is cis-3-OMe, R¹⁵ is trans-4-phosphate;

vi) where R¹ is OCH₃ in combination with R² is CH₃, R³ is OCH₃, R⁴ is OH, R⁵ is H, R⁶ is H, R⁷ is H, R⁸, R⁹ is keto, R¹⁰ is H, X is CH₂, R is formula C, Y is a bond, R¹⁴ is cis-3-OH, R¹⁵ is trans-4-OH (41-desmethyl rapamycin);

vii) where R¹ is OCH₃ in combination with R² is CH₃, R³ is OH, R⁴ is OH, R⁵ is H, R⁶ is H, R⁷ is H, R⁸, R⁹ is keto, R¹⁰ is H, X is CH₂, R is formula C, Y is a bond, R¹⁴ is cis-3-OMe, R¹⁵ is trans-4-OH (32-desmethyl rapamycin);

viii) where R¹ is OH in combination with R² is CH₃, R³ is OCH₃, R⁴ is OH, R⁵ is H, R⁶ is H, R⁷ is H, R⁸,R⁹ is keto, R¹⁰ is H, X is CH₂, R is formula C, Y is a bond, R¹⁴ is cis-3-OMe, R¹⁵ is trans-4-OH (7-desmethylrapamycin);

ix) where R¹ is OCH₃ in combination with R² is CH₃, R³ is OCH₃, R⁴ is OH, R⁵ is H, R⁶ is H, R⁷ is H, R⁸, R⁹ is keto, R¹⁰ is H, X is bond, R is formula C, Y is a bond, R¹⁴ is cis-3-OMe, R¹⁵ is trans-4-OH (proline rapamycin)

In one embodiment, R¹⁴ and R¹⁵ are independently selected from among H, OH, halogen, thiol, amine, alkyl, an ester, an amide, a carbonate, a carbamate, a sulfonate, a phosphate, a tetrazole, and point of attachment to L.

The present invention further encompasses pharmaceutically acceptable salts and hydrates of the structure of formula (III).

When the compound of formula (III) is a member of a conjugate of the invention, at least one R¹, R³, R⁴, R¹⁴, R¹⁵ or R¹⁶ is a point of attachment of the linker to the rapalog core. In one embodiment, the point of attachment of the rapamycin to L is R¹ (42) or R³ (31).

A linker may be independently bound directly to any of these R groups. In another embodiment, the linker is bound through a bridging group selected from an alkyl, an ester, an ether, or a thioester, and a thioether.

In one embodiment, R is

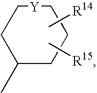

where Y is a bond or CHR¹⁶, R¹⁶ is selected from among H, alkyl, halogen, and hydroxyl, to provide a rapalog of formula (IIIa):

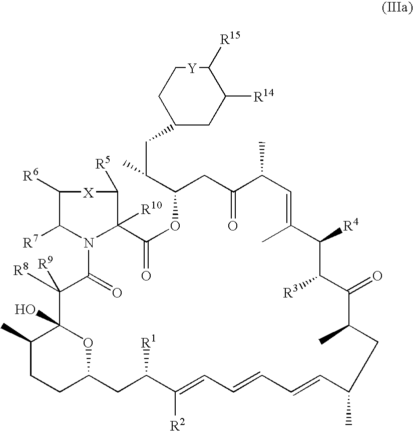

With the proviso that the compounds do not include the following:

i) where R¹ is OCH₃ in combination with R² is CH₃, R³ is OCH₃, R⁴ is OH, R⁵ is H, R⁶ is H, R⁷ is H, R⁸, R⁹ is keto, R¹⁰ is H, X is CH₂, Y is a bond, R¹⁴ is cis-3-OMe, R¹⁵ is trans-4-OH (rapamycin)

ii) where R¹ is OCH₃ in combination with R² is CH₃, R³ is OCH₃, R⁴ is OH, R⁵ is H, R⁶ is H, R⁷ is H, R⁸, R⁹ is keto, R¹⁰ is H, X is CH₂, Y is a bond, R¹⁴ is cis-3-OMe, R¹⁵ is a trans-4-ether such as trans-4-O—CH₂CH₂—OH (RAD001)

iii) where R¹ is OCH₃ in combination with R² is CH₃, R³ is OCH₃, R⁴ is OH, R⁵ is H, R⁶ is H, R⁷ is H, R⁸, R⁹ is keto, R¹⁰ is H, X is CH₂, Y is a bond, R¹⁴ is cis-3-OMe, R¹⁵ is a trans-4-ester such as trans-4-O—COC(CH₃)(CH₂OH)₂ (CCI779)

iv) where R¹ is OCH₃ in combination with R² is CH₃, R³ is OCH₃, R⁴ is OH, R⁵ is H, R⁶ is H, R⁷ is H, R⁸, R⁹ is keto, R¹⁰ is H, X is CH₂, Y is a bond, R¹⁴ is cis-3-OMe, R¹⁵ is cis-4-tetrazole (ABT578)

v) where R¹ is OCH₃ in combination with R² is CH₃, R³ is OCH₃, R⁴ is OH, R⁵ is H, R⁶ is H, R⁷ is H, R⁸, R⁹ is keto, R¹⁰ is H, X is CH₂, Y is a bond, R¹⁴ is cis-3-OMe, R¹⁵ is trans-4-phosphate.

In a further embodiment, R is

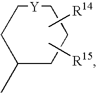

R¹ is OMe, R⁵ is H, R⁶ is H, R⁷ is H, R⁸, R⁹ are keto, R¹⁰ is H, X is a bond or CH₂, Y is a bond or CH₂, to provide a rapalog of formula (IIIb):

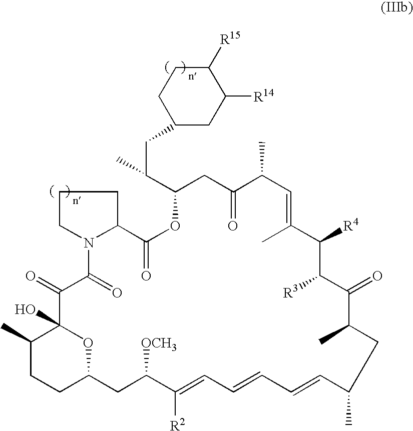

Wherein n′ and n″ are independently selected from among 1 or 2; and all other groups are as defined above.

In a still further embodiment, n′ is 1, n″ is 2, R⁴ is OH, R¹⁵ is trans-4-OH to provide the rapalog of formula (IIIc):

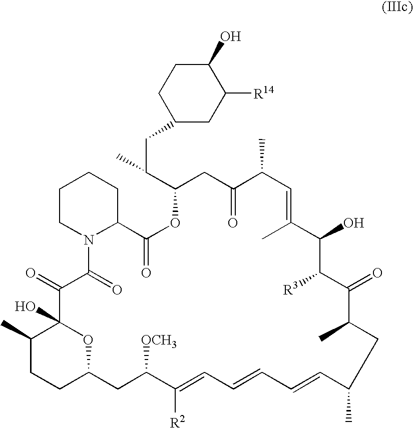

With the proviso that the compounds do not include R² is CH₃, R³ is OCH₃, and R¹⁴ is cis-3-OMe.

In still another embodiment, the rapalog is a 41-Desmethoxyrapamycin, in which R² is Me, R³ is OMe, and R¹⁴ is H, of formula (IIId):

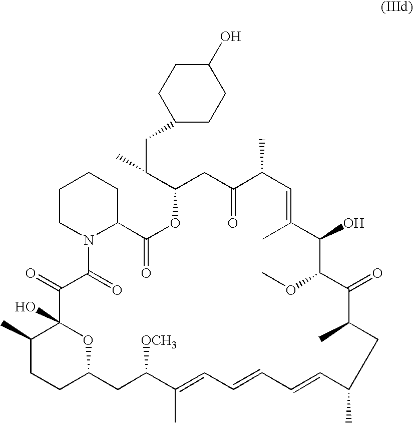

Methods of producing the compounds of formula (III) have been described. See, e.g., International Patent Publication Nos. WO 2004/007709, published 22 Jan. 2004; WO 2006/016167, published 16 Feb. 2006, US Published Patent Application Nos. 2005/0272132; 2006/0078980 and the documents cited therein. Other methods for generating such compounds will be readily apparent to one of skill in the art.

As defined above, the term “a rapalog” includes esters, ethers, amides, carbonates, carbamates, sulfonates, phosphates, tetrazines, oximes, hydrazones, and hydroxyamines of formula (III), including e.g., formula (IIIa), formula (IIIb), formula (IIIc), and formula (IIId), in which functional groups on the nucleus have been modified, for example through reduction or oxidation, replacement with a nucleophile such as tetrazole, a metabolite of rapalog such as various desmethylrapamycin derivatives or a ring opened rapalog. The term rapalog also includes pharmaceutically acceptable salts of the structures defined herein, which are capable of forming such salts, either by virtue of containing an acidic or basic moiety.

The term “desmethoxyrapamycin” and “desmethoxyrapalog” refers to a rapamycin or a rapalog nucleus lacking one or more methoxy groups (i.e., having an H bound to the C, missing OMe). In one embodiment, the rapalog is a 41-desmethoxyrapamycin as shown in formula (IIId).

The terms “desmethylrapamycin” and “O-desmethylrapamycin” are used interchangeably throughout the literature and the present specification, unless otherwise specified. The term “desmethylrapamycin” refers to the class of immunosuppressive compounds which contain the basic rapamycin nucleus shown, but lacking one or more methyl groups. In one embodiment, the rapamycin nucleus is missing a methyl group from either positions 7, 32, or 41, or combinations thereof. The synthesis of other desmethylrapamycins may be genetically engineered so that methyl groups are missing from other positions in the rapamycin nucleus. Production of desmethylrapamycins has been described. See, e.g., 3-desmethylrapamycin [U.S. Pat. No. 6,358,969], and 17-desmethylrapamycin [U.S. Pat. No. 6,670,168].

The term “desmethylrapalog” refers to the class of immunosuppressive compounds which contain the basic rapalog nucleus shown in formula (III), including e.g., formula (IIIa), formula (IIIb), formula (IIIc), and formula (IIId), but lacking one or more methyl groups.

Suitably, the rapalogs described herein exclude rapamycin, CCI-779, proline rapamycin, RAD001 (everolimus, Novartis), ABT578 (Abbott), and/or AP23573 [Ariad]. In one embodiment, the rapalogs useful in this invention exclude compounds of formula (III), including (IIIa)-(IIId), wherein R is

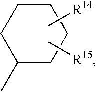

when X″ is CH₂, R⁸/R⁹ is ═O and H, R¹⁴ is selected from among OH, ester, ether, and amide, and R¹⁵ is selected from among OH, ester, ether, amide, carbonate, carbamate, phosphate and tetrazole.

Unless otherwise specified, an “amide” is —CONH—, where the carbon atom is generally bound to a hydrocarbon radical and the N forms the point of attachment to the rapalog core.

A “carbonate” contains a —OC(O)O— group. One oxygen atom is generally bound to a hydrocarbon radical, and the other oxygen atom forms the point of attachment to the rapalog core.

A “carbamate” contains a —NH(CO)O— group, where either nitrogen or oxygen is generally bound to a hydrocarbon radical. In one embodiment, O or N forms the point of attachment to the rapalog core.

A “sulfonate” contains a —S(O)₂O— group, where the S atom is generally bound to a hydrocarbon radical. The O forms the point of attachment to the rapalog core.

A “phosphate” contains a —OP(O)(OR)₂— group, where the O forms the point of attachment to the rapalog core and R is generally a hydrocarbon radical.

An “ether” has the structure —O—, where one group on the oxygen is generally a hydrocarbon radical. The O forms the point of attachment to the rapalog core.

An “ester” has the structure —C(O)O—, where the carbon atom is generally bound to a hydrocarbon radical and the O forms the point of attachment to the rapalog core.

As used herein, pharmaceutically acceptable salts include, but are not limited to, hydrochloric, hydrobromic, hydroiodic, hydrofluoric, sulfuric, citric, maleic, acetic, lactic, nicotinic, succinic, oxalic, phosphoric, malonic, salicylic, phenylacetic, stearic, pyridine, ammonium, piperazine, diethylamine, nicotinamide, formic, urea, sodium, potassium, calcium, magnesium, zinc, lithium, cinnamic, methylamino, methanesulfonic, picric, tartaric, triethylamino, dimethylamino, and tris(hydroxymethyl)aminomethane. Additional pharmaceutically acceptable salts are known to those skilled in the art.

A wortmannin according to the present invention refers to wortmannin and to compounds which may be chemically or biologically modified as derivatives of the wortmannin nucleus, while retaining biological activity. Accordingly the term “a wortmannin” includes wortmannin and esters, ethers, oximes, hydrazones, and hydroxyamines of wortmannin, as well as wortmannins in which functional groups on the nucleus have been modified, for example through reduction or oxidation, a metabolite of wortmannin or a ring opened wortmannin. The term wortmannin also includes pharmaceutically acceptable salts of wortmannins, which are capable of forming such salts, either by virtue of containing an acidic or basic moiety. See, e.g., U.S. Pat. No. 5,378,725.

In one embodiment, a “wortmannin” is characterized by the class of compounds having the core structure (Ia) provided below:

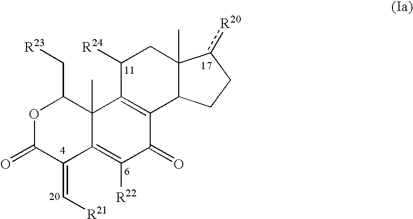

wherein, R²⁰ is selected from among O, OH, an ester, a carbonate, a carbamate, and an ether; R²¹ and R²² are bound together via an O heteroatom, or R²¹ is selected from among an ester, an ether, a thioether, a thioester, and an amino group; and R²² is selected from among OH, an ester, a carbonate, a carbamate, an ether, and a thioether; R²³ is selected from among OH, an ester, and an ether; R²⁴ is selected from among O, OH, an ester, a carbonate, and a carbamate.

When in the form of a conjugate of the invention, at least one of R²⁰, R²¹, R²², R²³, and R²⁴ is a point of attachment to the linker (L). In one embodiment, the point of attachment of the wortmannin to L is R²⁰ (17′) or R²⁴ (11′).

A linker may be independently bound directly to any of the R²⁰-R²⁴ groups or bound through a bridging group. Such a bridging group may be independently selected from the groups recited above for R²⁰-R²⁴. In other embodiments, the bridging group may be selected from an alkyl, an ester, an ether, or a thioester, and a thioether.

In one embodiment, R²⁰ is O, R²⁴ is OAc, and R²¹ and R²² are bound together via an O heteroatom to form the wortmannin core. In another embodiment, R²¹ is selected from among diethylamine, diallylamine, N,N,N′-trimethyl-1,3-propanediamine, piperidine, and N,N-dimethyl-N′-ethyl-ethylenediamine and R¹³ is —OH.

In one embodiment, the wortmannin may be a ring-opened wortmannin in which the furan ring is opened, i.e., R²¹ and R²² are independent substituents. Nucleophilic addition to the electrophilic C-20 position of wortmannin results in a wortmannin derivative in which the furan ring is opened. Such ring-opened compounds are described in (Wipf, Peter, et al., (2004) “Synthesis and biological evaluation of synthetic viridins derived from C(20)-heteroalkylation of the steroidal PI-3-kinase inhibitor wortmannin,” Org. Biomol. Chem., 2, 1911-1920); US Patent Application Publication No. 2003/0109572 to Powis; US Patent Application Publication No. 2006-0128793 (application Ser. No. 11/248,510, filed Oct. 10, 2005).

In another embodiment, the wortmannin is a 17-hydroxywortmannin (Ia), wherein the linker (L) is attached through the group at the 17-position of wortmannin, (R²⁰) and R²¹, R²², R²³, and R²⁴ are as defined above.

17-Hydroxywortmannins may be prepared by the reduction of wortmannin, for example with diborane. 17-Hydroxywortmannins and other derivatives may be prepared according to US Patent Application Publication No. 2004/0213757 and US Published Patent Application No. 2006-0128793. Further wortmannin derivatives may be derived form the acetylation of C-17 hydroxyl group. 17-hydroxywortmannin can be treated with a nucleophile such as an amine to give a furan ring opened compound. 17-hydroxywortmannin can also be formulated at the 17-position then treated with a nucleophile to give a furan ring opened compound.

In another embodiment, 11-O-desacetylwortmannin may be prepared by the literature procedure (Creemer C. L., et al., (1996), “Synthesis and in vitro Evaluation of New Wortmannin Esters: Potent Inhibitors of Phosphatidylinositol 3-Kinase”, J. Med. Chem. 39, 5021-5024). Further 11-O-desacetylwortmannin derivatives are furan ring opened compounds with nucleophiles.

In another embodiment, the wortmannin may be conjugated to a water-soluble polymer such as PEG and as described in US Patent Application Publication Nos. 2004/0213757 and 2006-0128793 (Ser. No. 11/248,510, filed Oct. 13, 2005).

The biosynthetic production of wortmannin is known in the art and the derivatives are synthesized from wortmannin.

### Conjugates

The invention provides a conjugate formed by linking a rapalog and a wortmannin via a linker. Suitably, following administration of a conjugate to a subject, the linker is removed from one or both of the rapalog or the wortmannin.

The linker may be removed by any process without limitation, e.g., hydrolysis, enzymatic, pH, etc. In one embodiment, the linker is hydrolysable. In another embodiment, the linker is enzymatically cleaved. The term “hydrolysed” or “hydrolysable” and “enzymatically cleaved” or “enzymatically cleavable” as used herein refers to the mechanism by which the linker group is released in vivo.

The linker may be completely removed from one or both of its binding partners (i.e., the rapalog or the wortmannin) following administration of the conjugate to a subject. In such an embodiment, no member of the linker group remains bound to the rapalog or the wortmannin following its removal. In another embodiment, the linker is partially removed from one or both of its binding partners following administration of the conjugate to a subject. In this embodiment, the linker is cleaved such that the rapalog and the wortmannin are separated; however, some part of the linker remains bound to the rapalog or wortmannin. In one embodiment, a composition comprising an effective amount of conjugates may be processed in vivo, such that the conjugates afford a mixture of partially and completely removed linker—rapalog and/or partially and completely removed linker—wortmannin metabolites.

In one embodiment, the linker is characterized by formula (V):

-Z¹-X-Z²- (V)

wherein, Z¹ and Z² are independently selected from among a bond, —O—, —N(R⁰)—, —S—, —OC(═O)—, —OC(═O)O—, —N(R⁰)C(═O)—, —OC(═O)N(R⁰)—, —N(R⁰)C(═O)N(R⁰)—, —OC(═S)N(R⁰)—, —N(R⁰)C(═S)N(R⁰)—, and ═N—N(R⁰)—; R⁰ is at each occurrence independently selected from among H, alkyl, alkenyl, and aryl; and X is selected from among a hydrocarbon chain having 1 to 16 carbon atoms which may be branched or unbranched, saturated or unsaturated, and optionally substituted with one or more of oxy, amine, sulfide, alkyl, alkenyl, aryl, alkoxy, hydroxyl, and halogen and/or interrupted by one or more ether (—O—), amine (—NH—) or sulfide (—S—), —S(O)ₙ—, NR⁰, —C(═O)N(R⁰)—, —OC(═O)N(R⁰)—, —N(R⁰)C(═O)N(R⁰)—, —OC(═S)N(R⁰)—, —N(R⁰)C(═S)N(R⁰)—, or ═N—N(R⁰)—, or a combination thereof. X may also be selected from among a cycloalkyl, alkylarylalkyl, heteroaryl, and a heterocyclic group. In one embodiment, Z is a bond, i.e., L is -Z¹-X—, —X—, or —X-Z²-.

In one embodiment, the conjugate excludes peroxide (O—O), O—N, and O—S bonds between the rapalog or wort core and linker. Thus, the linker contains a terminal O, N or S group and the rapalog or wort core does not provide for an O to be bound to the group.

As used herein, the term “alkyl” refers to both straight- and branched-chain saturated aliphatic hydrocarbon groups. In one embodiment, an alkyl group has 1 to about 16 carbon atoms. In another embodiment, an alkyl group has 1 to 10 carbon atoms or 1 to 8 carbon atoms (i.e., C₁, C₂, C₃, C₄, C₅ C₆, C₇, C₈). An alkyl group having 1 to about 6 carbon atoms (i.e., C₁, C₂, C₃, C₄, C₅ or C₆) may be referred to as a “lower alkyl” group. In a further embodiment, an alkyl group has 1 to about 4 carbon atoms (i.e., C₁, C₂, C₃, or C₄). Particularly desirable alkyl groups include methyl, ethyl, propyl, isopropyl, butyl, isobutyl, and tert-butyl. Unless other substituents are specified, the alkyl group may be optionally substituted with one or more substituents selected from halo, CN, CO₂R, C(O)R, C(O)NR₂, NR₂, NO₂, and OR. Other suitable substituents are described herein in the definition of “substituted alkyl”.

The term “alkylarylalkyl” or “alkylaralkyl” refers to an alkyl group which is substituted with an aryl group which is itself substituted with an alkyl group.

The term “aryl” as used herein refers to an aromatic, carbocyclic system, e.g., of about 4 to 14 carbon atoms, which can include a single ring or multiple aromatic rings fused or linked together where at least one part of the fused or linked rings forms the conjugated aromatic system. The aryl groups include, but are not limited to, phenyl, naphthyl, biphenyl, anthryl, tetrahydronaphthyl, phenanthryl, indene, benzonaphthyl, and fluorenyl.

The term “cycloalkyl” is used herein to refer to cyclic, saturated aliphatic hydrocarbon groups. In one embodiment, a cycloalkyl group has 3 to about 8 carbon atoms (i.e., C₃, C₄, C₅, C₆, C₇, or C₈). In another embodiment, a cycloalkyl group has 3 to about 6 carbon atoms (i.e., C₃, C₄, C₅ or C₆).

The term “alkoxy” as used herein refers to the O(alkyl) group, where the point of attachment is through the oxygen-atom and the alkyl group can be substituted as noted above.

The term halo, or halogen, refers to elemental Cl, Br, F, or I or a group containing same.

The term “heterocycle” or “heterocyclic” as used herein can be used interchangeably to refer to a stable, saturated or partially unsaturated 3- to 9-membered monocyclic or multicyclic heterocyclic ring. The heterocyclic ring has in its backbone carbon atoms and one or more heteroatoms including nitrogen, oxygen, and sulfur atoms. In one embodiment, the heterocyclic ring has 1 to about 4 heteroatoms in the backbone of the ring. When the heterocyclic ring contains nitrogen or sulfur atoms in the backbone of the ring, the nitrogen or sulfur atoms can be oxidized. The term “heterocycle” or “heterocyclic” also refers to multicyclic rings in which a heterocyclic ring is fused to an aryl ring of about 6 to about 14 carbon atoms. The heterocyclic ring can be attached to the aryl ring through a heteroatom or carbon atom provided the resultant heterocyclic ring structure is chemically stable. In one embodiment, the heterocyclic ring includes multicyclic systems having 1 to 5 rings.

A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Examples of heterocyclic groups include, without limitation, tetrahydrofuranyl, piperidinyl, 2-oxopiperidinyl, pyrrolidinyl, morpholinyl, thiamorpholinyl, thiamorpholinyl sulfoxide, pyranyl, pyronyl, dioxinyl, piperazinyl, dithiolyl, oxathiolyl, dioxazolyl, oxathiazolyl, oxazinyl, oxathiazinyl, benzopyranyl, benzoxazinyl and xanthenyl.

The term “heteroaryl” as used herein refers to a stable, aromatic 5- to 14-membered monocyclic or multicyclic heteroatom-containing ring. The heteroaryl ring has in its backbone carbon atoms and one or more heteroatoms including nitrogen, oxygen, and sulfur atoms. In one embodiment, the heteroaryl ring contains 1 to about 4 heteroatoms in the backbone of the ring. When the heteroaryl ring contains nitrogen or sulfur atoms in the backbone of the ring, the nitrogen or sulfur atoms can be oxidized. The term “heteroaryl” also refers to multicyclic rings in which a heteroaryl ring is fused to an aryl ring. The heteroaryl ring can be attached to the aryl ring through a heteroatom or carbon atom provided the resultant heterocyclic ring structure is chemically stable. In one embodiment, the heteroaryl ring includes multicyclic systems having 1 to 5 rings.

A variety of heteroaryl groups are known in the art and include, without limitation, oxygen-containing rings, nitrogen-containing rings, sulfur-containing rings, mixed heteroatom-containing rings, fused heteroatom containing rings, and combinations thereof. Examples of heteroaryl groups include, without limitation, furyl, pyrrolyl, pyrazolyl, imidazolyl, triazolyl, pyridyl, pyridazinyl, pyrimidinyl, pyrazinyl, triazinyl, azepinyl, thienyl, dithiolyl, oxathiolyl, oxazolyl, thiazolyl, oxadiazolyl, oxatriazolyl, oxepinyl, thiepinyl, diazepinyl, benzofuranyl, thionapthene, indolyl, benzazolyl, purindinyl, pyranopyrrolyl, isoindazolyl, indoxazinyl, benzoxazolyl, quinolinyl, isoquinolinyl, benzodiazonyl, napthylridinyl, benzothienyl, pyridopyridinyl, acridinyl, carbazolyl, and purinyl rings.

The term “substituted heterocycle” and “substituted heteroaryl” as used herein refers to a heterocycle or heteroaryl group having one or more substituents including halogen, CN, OH, NO₂, amino, alkyl, cycloalkyl, alkenyl, alkynyl, C₁ to C₃ perfluoroalkyl, C₁ to C₃ perfluoroalkoxy, alkoxy, aryloxy, alkyloxy including —O—(C₁ to C₁₀ alkyl) or —O—(C₁ to C₁₀ substituted alkyl), alkylcarbonyl including —CO—(C₁ to C₁₀ alkyl) or —CO—(C1 to C₁₀ substituted alkyl), alkylcarboxy including —COO—(C₁ to C₁₀ alkyl) or —COO—(C₁ to C₁₀ substituted alkyl), —C(NH₂)═N—OH, —SO₂—(C₁ to C₁₀ alkyl), —SO₂—(C₁ to C₁₀ substituted alkyl), —O—CH₂-aryl, alkylamino, arylthio, aryl, substituted aryl, heteroaryl, or substituted heteroaryl which groups may be optionally substituted. A substituted heterocycle or heteroaryl group may have 1, 2, 3, or 4 substituents.

The term “alkenyl” is used herein to refer to both straight- and branched-chain alkyl groups having one or more carbon-carbon double bonds. In one embodiment, an alkenyl group contains 2 to about 8 carbon atoms (i.e., C₂, C₃, C₄, C₅, C₆, C₇, or C₈). In another embodiment, an alkenyl groups has 1 or 2 carbon-carbon double bonds and 3 to about 6 carbon atoms (i.e., C₃, C₄, C₅ or C₆).

The term “alkynyl” group is used herein to refer to both straight- and branched-chain alkyl groups having one or more carbon-carbon triple bonds. In one embodiment, an alkynyl group has 2 to about 8 carbon atoms (i.e., C₂, C₃, C₄, C₅, C₆, C₇, or C₈). In another embodiment, an alkynyl group contains 1 or 2 carbon-carbon triple bonds and 3 to about 6 carbon atoms (i.e., C₃, C₄, C₅, or C₆).

The terms “substituted alkyl”, “substituted alkenyl”, “substituted alkynyl”, and “substituted cycloalkyl” refer to alkyl, alkenyl, alkynyl, and cycloalkyl groups, respectively, having one or more substituents including, without limitation, hydrogen, halogen, CN, OH, NO₂, amino, aryl, heterocyclic groups, alkoxy, aryloxy, alkylcarbonyl, alkylcarboxy, amino, and arylthio.

In one embodiment, Z¹ and Z² are independently selected from a bond, —O—, —OC(═O)—, —OC(═O)O—, —OC(═O)N(R⁰)—, and OC(═S)N(R⁰)—.

In one embodiment, the conjugate excludes O—O, O—N and O—S linkages between the rapalog and the linker.

In one embodiment, Z¹ and Z² are a bond and X is an alkyl chain of 1 to 10 carbon atoms optionally substituted with one, two, or more O groups.

In one embodiment, X is independently selected from among C₁-C₈ alkyl, C₂-C₈ alkenyl, cycloalkyl, aryl, and a heterocyclic group. In another embodiment, X is selected from among an alkyl chain of 1 to 16 carbon atoms interrupted by at least one group selected from —O—, —S(O)ₙ—, —N(R⁰)—, —OC(═O)—, —OC(═O)O—, —C(═O)N(R⁰)—, and —OC(═O)N(R⁰)—, where n is 0 to 2. In one embodiment, X is (CH₂CH₂O)ₙ or —(CH₂)ₙ—O—(CH₂)ₙ—, where n is 1 to 8. In another embodiment, X is selected from among (CH₂)₂, (CH₂OCH₂), (CH₂)₃, (CH₂)₄, (CH₂)₅ and (CH₂)₆.

In one embodiment, the invention provides a rapalog covalently linked with a wortmannin through a dicarboxylic acid linker of the formula:

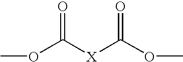

wherein, X is as defined above. For example, X may be a hydrocarbon chain of the formula —(CH₂)ₙ—, where n is 1 to 16. Alternatively, X may be a hydrocarbon chain interrupted by an ether linkage, having the formula —(CH₂)ₙ—O—(CH₂)ₙ—, where n is 1 to 8.

A linker may be bound independently to the rapalog nucleus (III) directly or through one of the selected groups of R¹, R³, R⁴, R¹⁴ or R¹⁵ groups. Alternatively, the linker may be bound to the rapalog core through a bridging group independently selected from among an alkyl, an oxime, a hydrazone, a hydroxylamine, an ester, an ether, a thioester, and a thioether.

In one embodiment, a conjugate contains a rapalog and a wortmannin in a ratio of 1:1, i.e., one rapalog linked to one wortmannin.

In one embodiment, the conjugate contains a rapalog nucleus of formula (III):

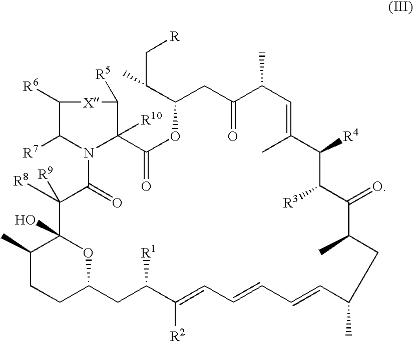

wherein, R¹ is selected from among OH, an ester, an ether, and a point of attachment to the linker, which linker may be bound to the core through one of the preceding groups; R² is methyl or H; R³ is selected from among H, OH, an ester, an ether, and a point of attachment to the linker, which linker may be bound to the core through one of the preceding groups; R⁴ is selected from among OH, an ester, an ether, an amide, a carbonate, a carbamate, a phosphate, and a point of attachment to the linker, which linker may be bound to the core through one of the preceding groups; R⁵, R⁶, and R⁷ are independently selected from among H, alkyl, halo, hydroxyl; R⁸, R⁹ is H, H or ═O; R¹⁰ is selected from among H, alkyl, halo or a hydroxyl; X″ is a bond or CHR¹¹; or —CHR⁵—X″—CHR⁶— is:

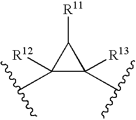

R¹¹, R¹², and R¹³ are independently selected from among H, alkyl, halo, and hydroxyl; R is selected from among:

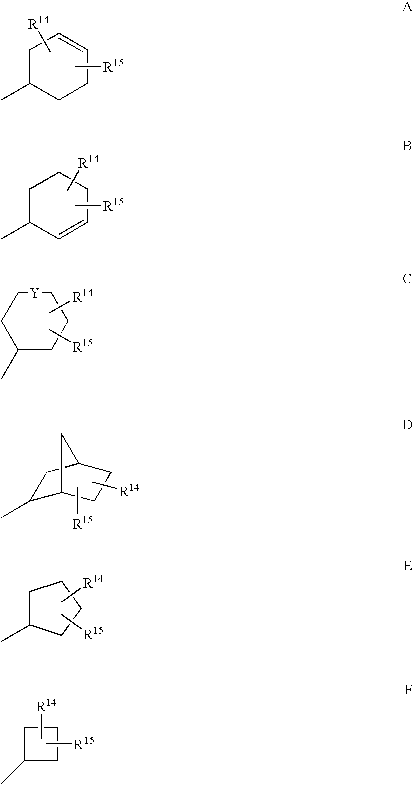

R¹⁴ and R¹⁵ are independently selected from H, OH, halogen, thiol, amine, alkyl, an ester and an ether, an amide, a carbonate, a carbamate, a sulfonate, a phosphate, a tetrazole, and a point of attachment to the linker, which linker may be bound to the core through one of the preceding groups; Y is a bond or CHR¹⁶, R¹⁶ is selected from among H, alkyl, halogen, hydroxyl, and a point of attachment to the linker, which linker may be bound to the core through one of the preceding groups.

In one embodiment, the rapalog of formula (III) excludes rapamycin, proline rapamycin, CCI-779, RAD001, ABT578 (Abbott), and/or AP23573 [Ariad].

In a further embodiment, the linker is independently bound directly to any of the R²⁰-R²⁴ groups of the wortmannin core (I) or through the selected group for one or more of these substituents. In still another alternative, the linker may be bound to the wortmannin core through a group independently selected from among an alkyl, an ester, an ether, or a thioester, and a thioether.

Thus, the wortmannin core of the conjugate is characterized by the formula (Ia):

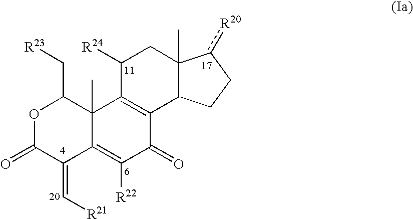

wherein, R²⁰ is selected from among O, OH, an ester, a carbonate, a carbamate, an ether, and a point of attachment to the linker, where the linker is optionally bound to the core through the selected group; R²¹ and R²² are bound together via an O heteroatom, or R²¹ is selected from among an ester, an ether, a thioether, a thioester, an amino group, and the point of attachment to the linker, wherein the linker is optionally bound to the core through the selected group and R²² is selected from the group selected from OH, an ester, a carbonate, a carbamate, an ether, a thioether, and a point of attachment to the linker, wherein the linker is optionally bound to the core through the selected group; R²³ is selected from among OH, an ester, an ether, and a point of attachment to the linker, wherein the linker is optionally bound to the core through the selected group; R²⁴ is selected from among O, OH, an ester, a carbonate, a carbamate, and a point of attachment to the linker, wherein the linker is optionally bound to the core through the selected group; wherein at least one of R²⁰, R²¹, R²², R²³, and R²⁴ is a point of attachment to the linker.

In one embodiment, the wortmannin nucleus may be further substituted at any of R²⁰-R²⁴ not bound to the linker, as described for the various wortmannin derivatives described above. For example, the wortmannin may be 17-hydroxywortmannin. In one embodiment, the linker is bound to the wortmannin nucleus through the 17-position. In another embodiment, a linker is bound to the 17-hydroxywortmannin through another position.

Examples of a variety of wortmannin-L-Rap formula are provided below:

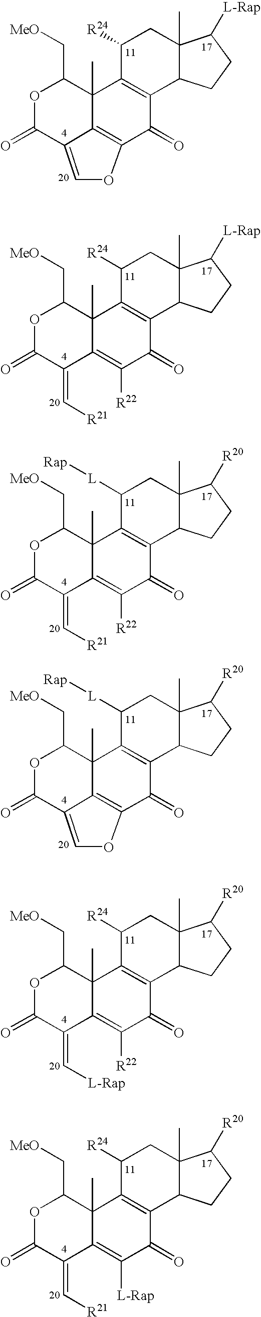

In one embodiment, R²⁰ is the point of attachment to the linker. In another embodiment, R²⁰ is an O.

In yet a further embodiment, R²¹ is an amino group. Desirably, where R²¹ is an amino group, it has the formula —NR^{a}R^{b}—, where R^{a} and R^{b} are independently selected from among H, alkyl, alkenyl, alkynyl, -(alkyl)-O-(alkyl)-, -(alkyl)-NR^{c}R^{d}—, -(alkyl)-C(═O)NR^{c}R^{d}—, cycloalkyl, aryl, heteroaryl, alkylaryl, alkylheteroaryl, and a heterocyclic group; or R^{a} and R^{b} may be taken together to form a three to seven membered heterocyclic ring having up to 3 heteroatoms which is optionally substituted by 1 to 3 substituents independently selected from among halogen, hydroxyl, thio, alkyl, alkenyl, alkoxy, oxo, amino, cyano, C₁-C₃ perfluoroalkyl, alkylaryl, alkylheteroaryl, aryl, and heteroaryl; R^{c} and R^{d} are independently selected from among H, alkyl, alkenyl, alkynyl, aryl, cycloalkyl, and heterocycyl; or R^{c} and R^{d} are taken together to form a three to seven membered cyclic or heterocyclic ring having up to 3 heteroatoms which is optionally substituted by 1 to 3 substituents independently selected from among halogen, hydroxyl, thio, alkyl, alkenyl, alkoxy, oxo, amino, cyano and C₁-C₃ perfluoroalkyl.

In one embodiment, R²¹ has the formula —NHR^{a}—, where R^{a} is as defined above. In one embodiment, R^{a} is phenyl.

In another embodiment R^{a} and R^{b} are lower alkyl.

In still another embodiment, R²¹ and R²² are bound together via an O heteroatom.

Other suitable points of attachment to wortmannins will be readily apparent to one of skill in the art.

### Methods of Preparing the Conjugates

The compounds described herein are readily prepared by one of skill in the art according to the following schemes using commercially available starting materials or starting materials which can be prepared using literature procedures. These schemes show the preparation of conjugates in which “a rapalog” is linked with “a wortmannin” through a di-ester linkage. While these schemes teach the principle of the present invention, with examples provided for the purpose of illustration, it will be understood that such conjugates through other types of functional group linkage such as amides, carbonates, carbamate, ether, thio, hydrazone, et al., or through other linking positions are readily available by modification of, or additions to, procedures and protocols described herein. Variations on these methods, or other methods known in the art, can be readily performed by one of skill in the art given the information provided herein.

In one embodiment, synthesis of rapalog-wortmannin conjugates 1 linked through rapalog (IIIc) 42-OH, and wortmannin 17-OH positions via a di-ester linkage, according to the present invention, is outlined in Scheme 1.

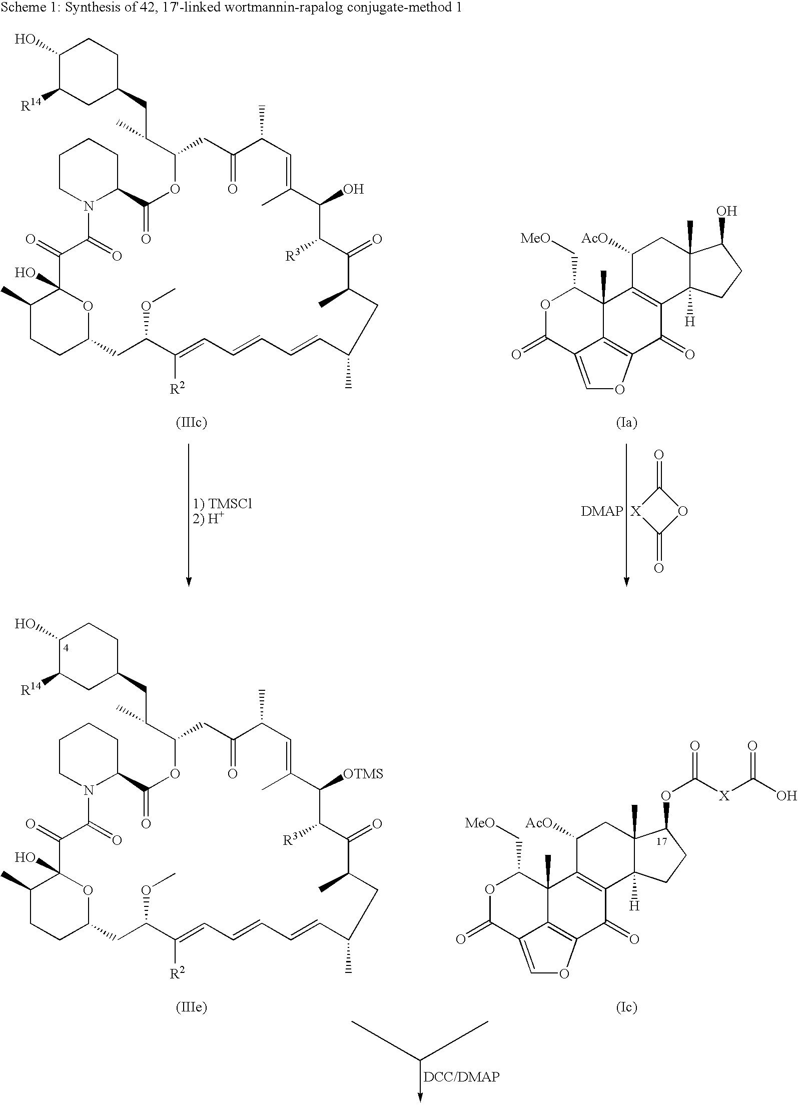

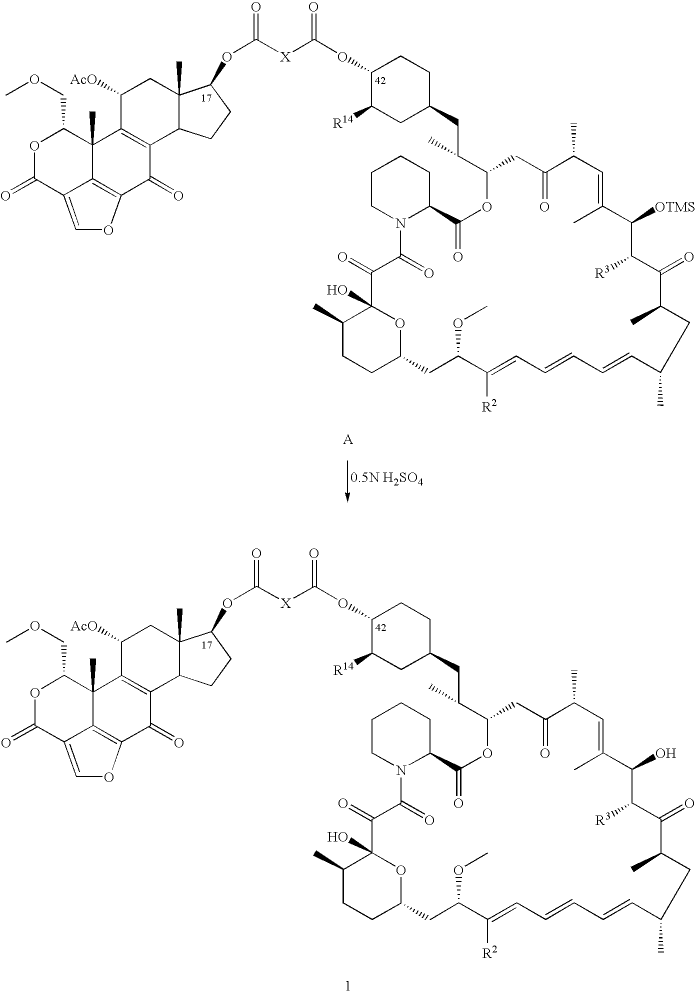

As outlined in Scheme 1, a 17-hydroxywortmannin (Ia) is acylated with various cyclic anhydrides to give hemiacids (Ic). These dicarboxylic monoesters are then coupled with rapalog 31-trimethylsilyl ether (IIIe) in the presence of a coupling reagent such as, e.g., N,N′-dicyclohexyl-carbodiimide (DCC), Diisopropylcarbodiimide (DIPC) or 1-Ethyl-3-[3-dimethylaminopropyl]-carbodiimide Hydrochloride (EDC) and a base such as 4-(dimethylamino)pyridine (DMAP), to give intermediates A. Subsequent de-protection with diluted H₂SO₄ furnish desired 42,17′-linked wortmannin-rapalog conjugate 1. Rapalog 31-trimethylsilyl ether (IIIe) may be synthesized according to the procedure described in U.S. Pat. No. 6,277,983.

Alternatively, such di-ester linked wortmannin-rapalog conjugates can be synthesized as described in Scheme 2. The dicarboxylic acid linker was first installed into rapalog's 42-OH via a lipase-catalyzed acylation method described in US Patent Application Publication No. 2005-0234087. These rapalog hemiesters (IIIf) were then coupled with 17-hydroxywortmannin (Ia) under DCC/DMAP combination to give 42,17′-linked wortmannin-rapalog conjugates 1 in good yield.

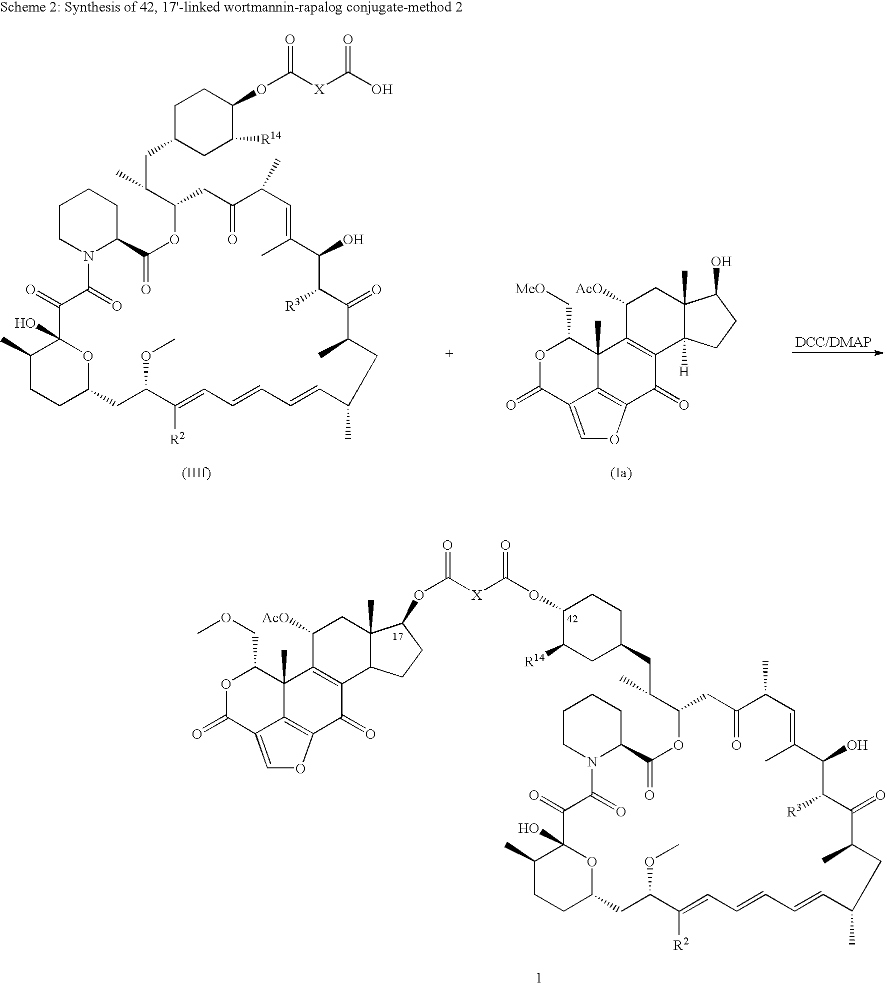

In one embodiment, exemplary conjugates which can be readily prepared using the techniques described herein include, e.g., those having the structures:

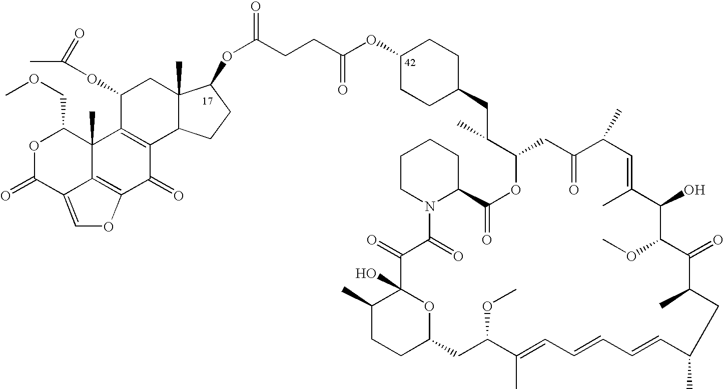

### 42,17′-linked wortmannin-succinate-41-desmethoxyrapamycin conjugate

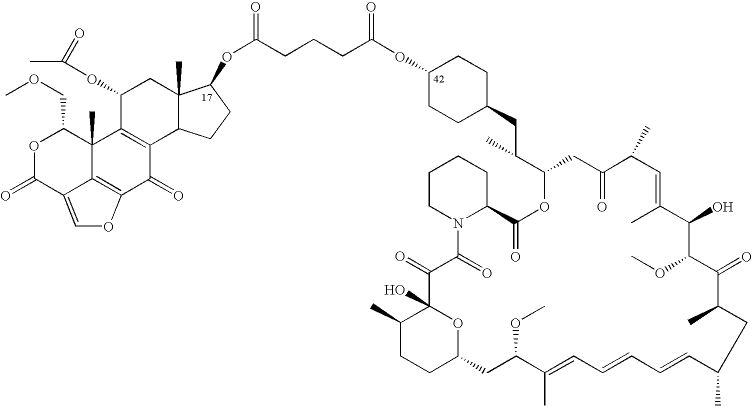

### 42,17′-linked wortmannin-glutarate-41-desmethoxyrapamycin conjugate

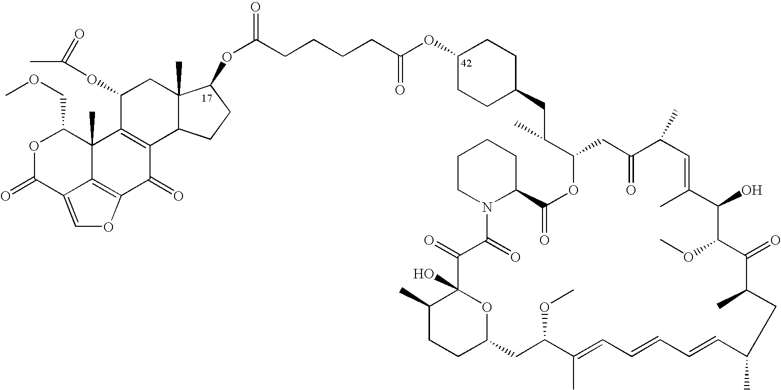

### 42,17′-linked wortmannin-adipate-41-desmethoxyrapamcyin conjugate

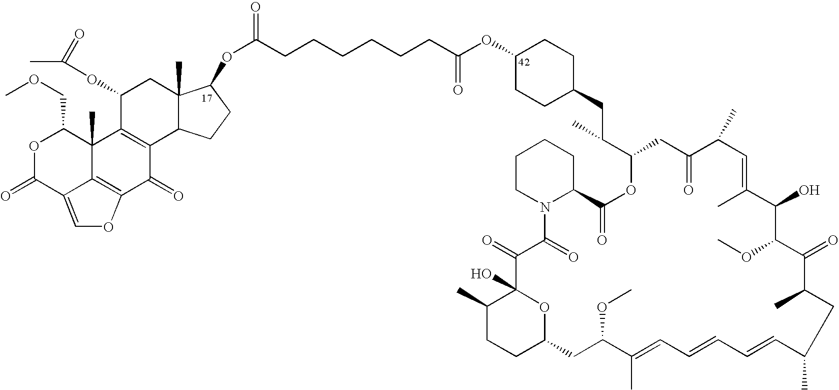

### 42,17′-linked wortmannin-suberate-41-desmethoxyrapamycin conjugate

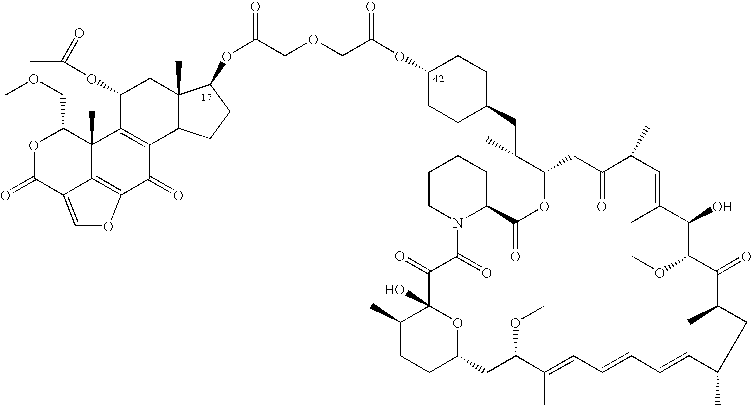

### 42,17′-linked wortmannin-diglycolinate-41-desmethoxyrapamycin conjugate

In one embodiment, these rapalog-wortmannin conjugates compounds can be further converted to the furan ring-opened derivatives 2 with various nucleophiles such as thiols, amines, particularly secondary amines, and alcohols (Scheme 3). These nucleophiles can be selected from among, for example, the list covered in US Published Patent Application No. 2006-0128793, published Jun. 15, 2006.

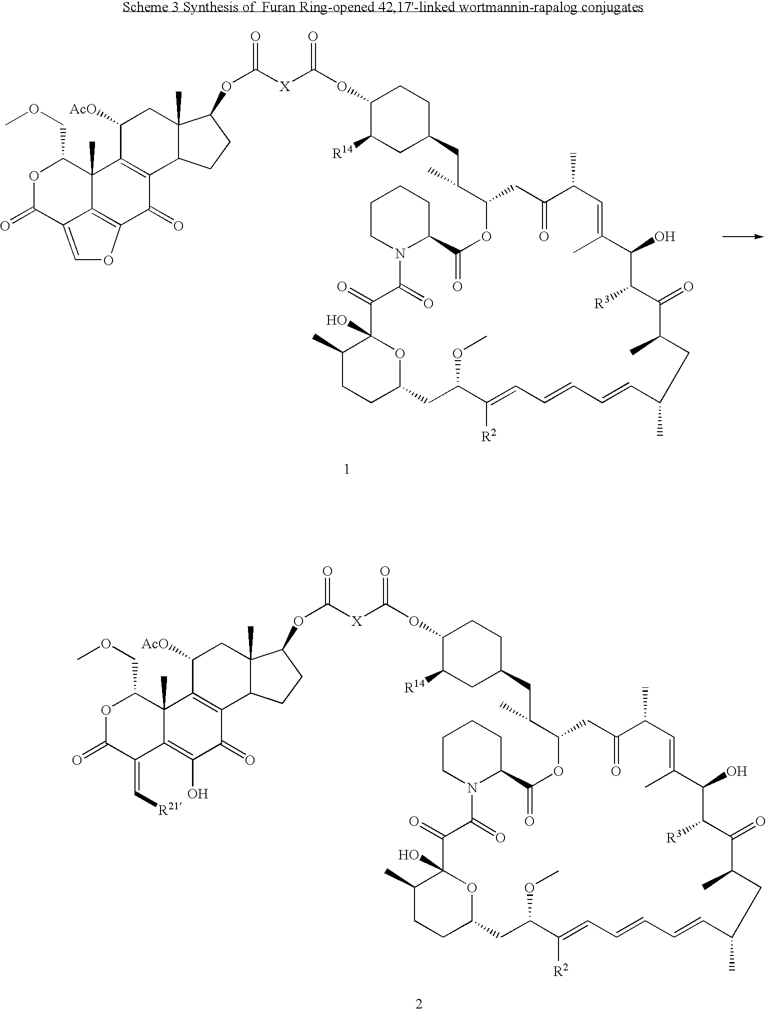

wherein, R^{21′} is selected from among NR^{a}R^{b}, SR^{c}, and OR^{d}, where R^{a} and R^{b} are independently selected from among H, alkyl, alkenyl, alkynyl, -(alkyl)-O-(alkyl), -(alkyl)-NR^{c}R^{d}, -(alkyl)-C(═O)NR^{c}R^{d}, cycloalkyl, aryl, heteroaryl, alkylaryl, alkylheteroaryl, and a heterocyclic group; or R^{a} and R^{b} may be taken together to form a three to seven membered heterocyclic ring having up to 3 heteroatoms which is optionally substituted by 1 to 3 substituents independently selected from among halogen, hydroxyl, thio, alkyl, alkenyl, alkoxy, oxo, amino, cyano, C₁-C₃ perfluoroalkyl, alkylaryl, alkylheteroaryl, aryl, and heteroaryl, R^{c} and R^{d} are independently selected from among H, alkyl, alkenyl, alkynyl, aryl, cycloalkyl, and heterocycyl; or R^{c} and R^{d} are taken together to form a three to seven membered cyclic or heterocyclic ring having up to 3 heteroatoms which is optionally substituted by 1 to 3 substituents independently selected from among halogen, hydroxyl, thio, alkyl, alkenyl, alkoxy, oxo, amino, cyano and C₁-C₃ perfluoroalkyl.

In one embodiment, exemplary conjugates in the form of amine adducts, i.e., furan ring in wortmannin was opened by various secondary amines, are illustrated below.

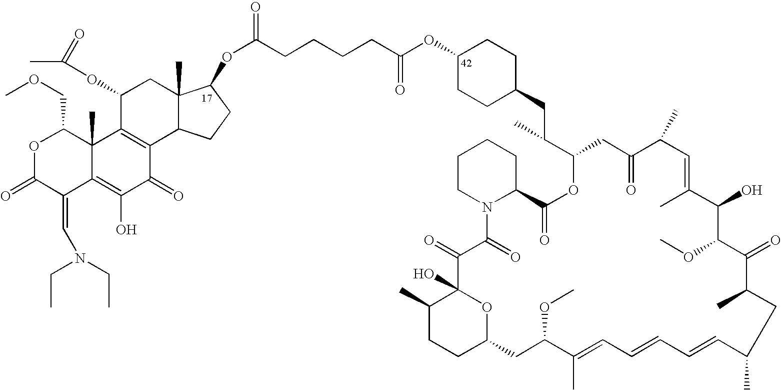

### 42,17′-linked wortmannin-adipate-41-desmethoxyrapamycin conjugate, diethylamine adduct

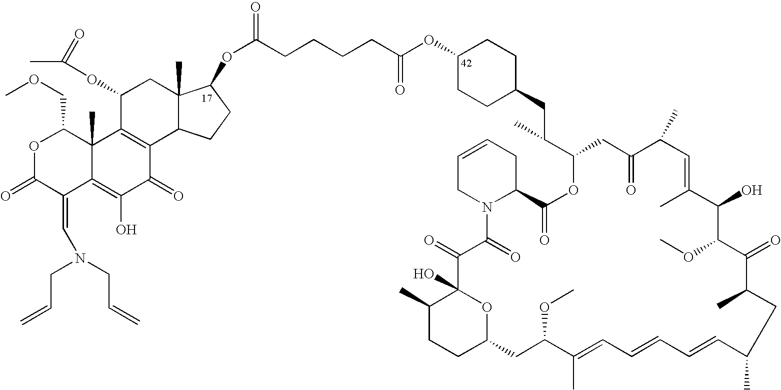

### 42,17′-linked wortmannin-adipate-41-desmethoxyrapamycin conjugate, diallylamine adduct

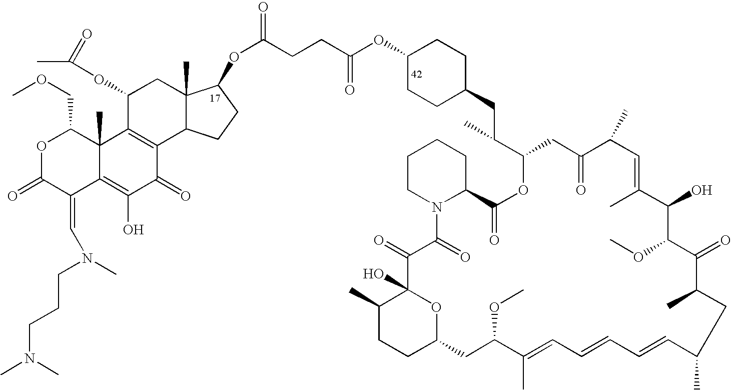

### 42,17′-linked wortmannin-succinate-41-desmethoxyrapamycin conjugate, N,N,N′-trimethyl 1,3-propanediamine adduct

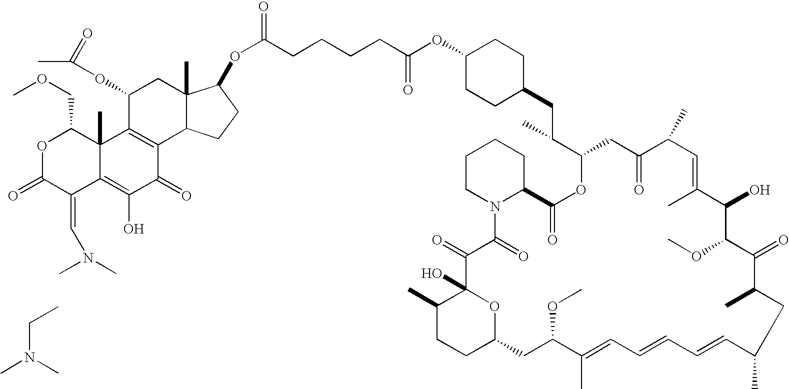

### 42,17′-linked wortmannin-adipate-41-desmethoxyrapamycin conjugate, N,N,N′-trimethyl 1,3-propanediamine adduct

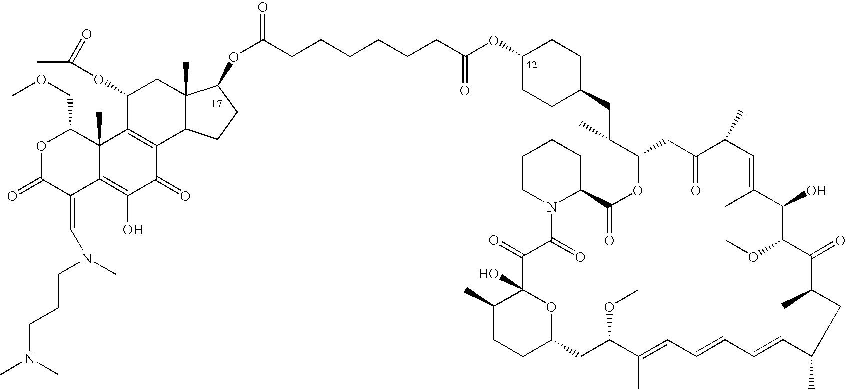

### 42,17′-linked wortmannin-suberate-41-desmethoxyrapamycin conjugate, N,N,N′-trimethyl 1,3-propanediamine adduct

In another embodiment, synthesis of rapalog-wortmannin conjugate through rapalog 31-OH and wortmannin 17-OH positions via a di-ester linkage is outlined in Scheme 4. The wortmannin 17-dicarboxylic monoacid (Ic) were coupled with rapalog 42-TBS ether (IIIg) in the presence of a coupling reagent such as, e.g., DCC, DIPC or EDC and a base such as DMAP, to give intermediates B. Subsequent de-protection with diluted H₂SO₄ furnishes the desired 31,17′-linked wortmannin-rapamycin conjugate 3. Rapamycin 42-TBS ether (IIIg) may be synthesized according to the procedure described in European Patent No. 0507556A1.

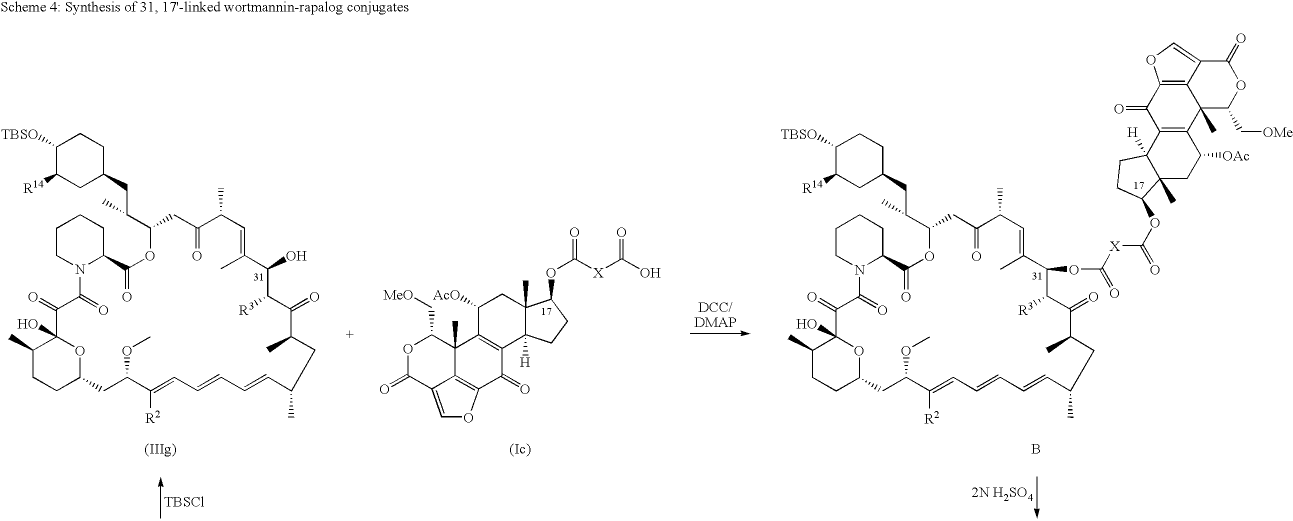

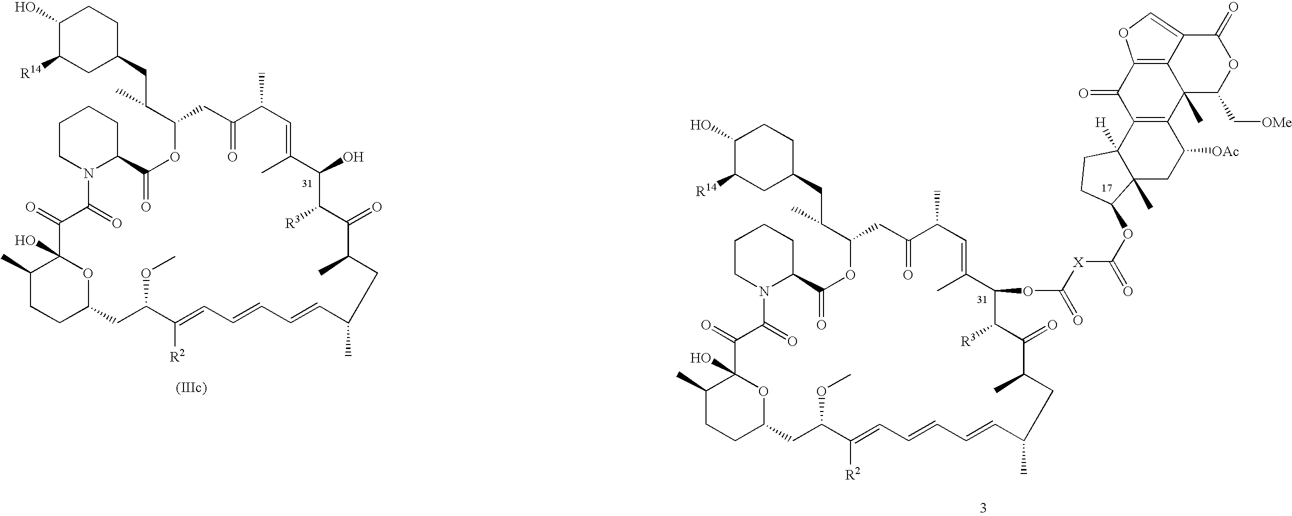

Illustrative examples according to the above preparation include, but are not limited to the following structure:

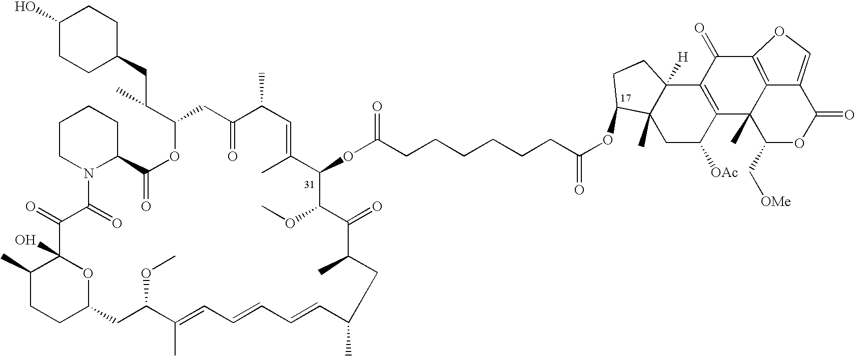

### 31,17′-linker wortmannin-suberate-41-desmethoxyrapamycin conjugate

In one embodiment, 31,17-linked wortmannin-rapalog conjugate 3 can be treated with R^{21′} containing nucleophiles to give a furan ring opened conjugate 4 as depicted in Scheme 5.

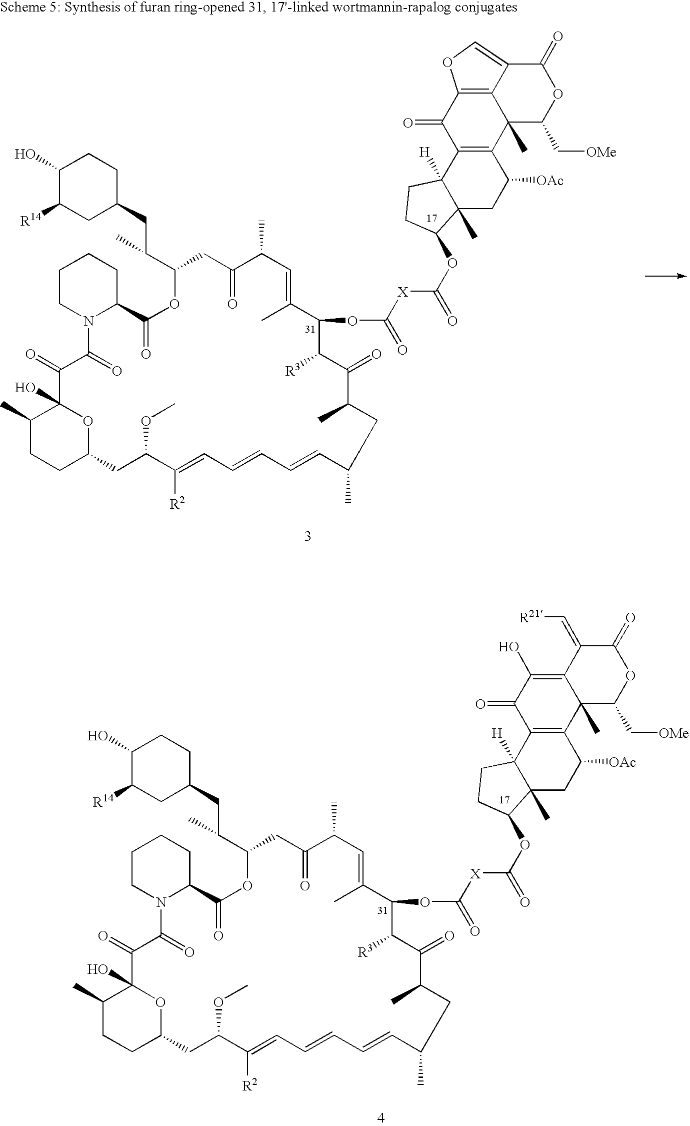

In one embodiment, the following exemplary compounds may be prepared:

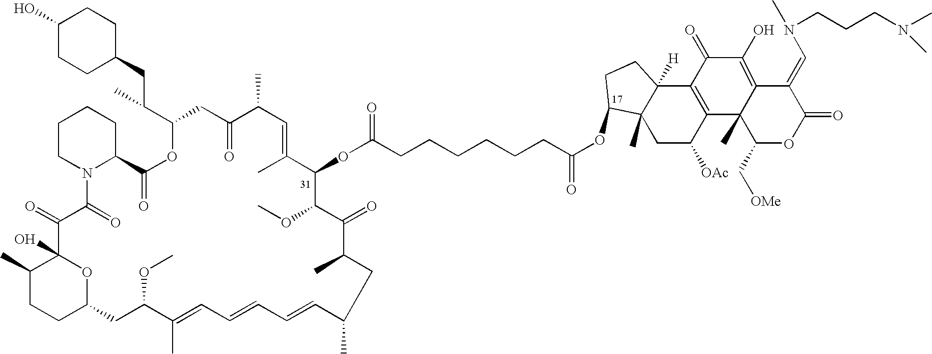

### 31,17-linked wortmannin-suberate-41-desmethoxyrapamycin conjugate, N,N,N′-trimethyl 1,3-propanediamine adduct

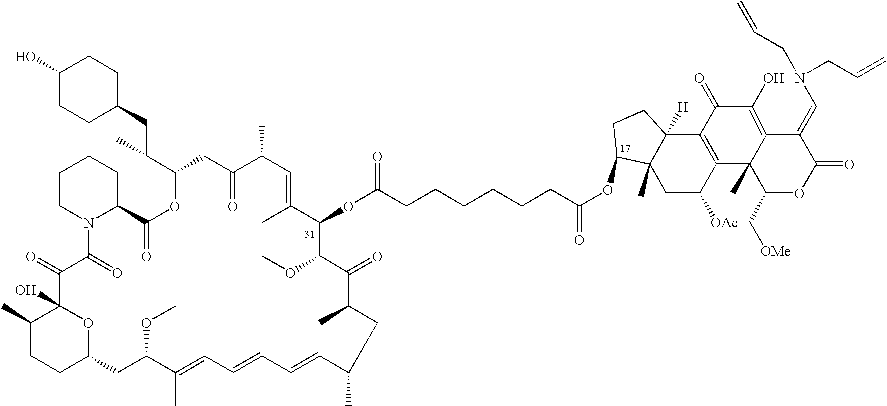

### 31,17′-linked wortmannin-suberate-41-desmethoxyrapamycin conjugate, diallylamine adduct

In still another embodiment, the conjugates can be prepared according to the Scheme 6 through the linking position of rapalog 42-OH and wortmannin 11-OH. As for di-ester linked wortmannin-rapalog, such conjugates (5) are readily available by coupling 11-desacetyl wortmannin 11-dicarboxylic monoacid (Id) with rapalog 31-TMS ether (IIIe) in the presence of a coupling reagent such as, e.g., DCC, DIPC or EDC and a base such as DMAP, followed by de-protection with diluted H₂SO₄ in good overall yield.

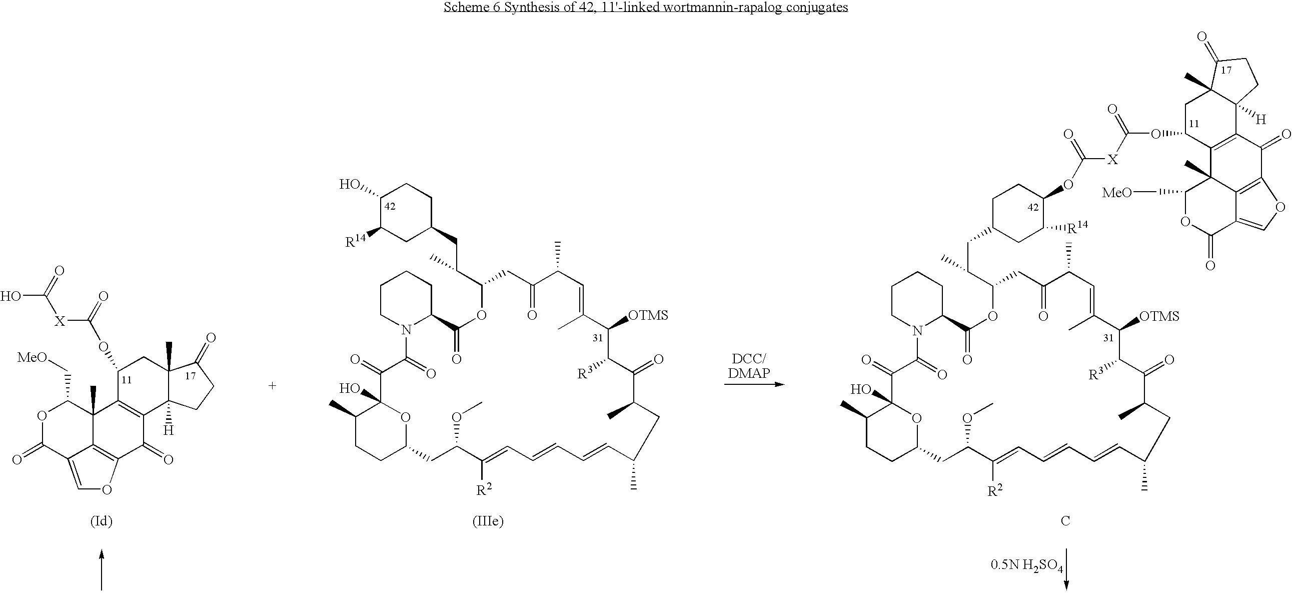

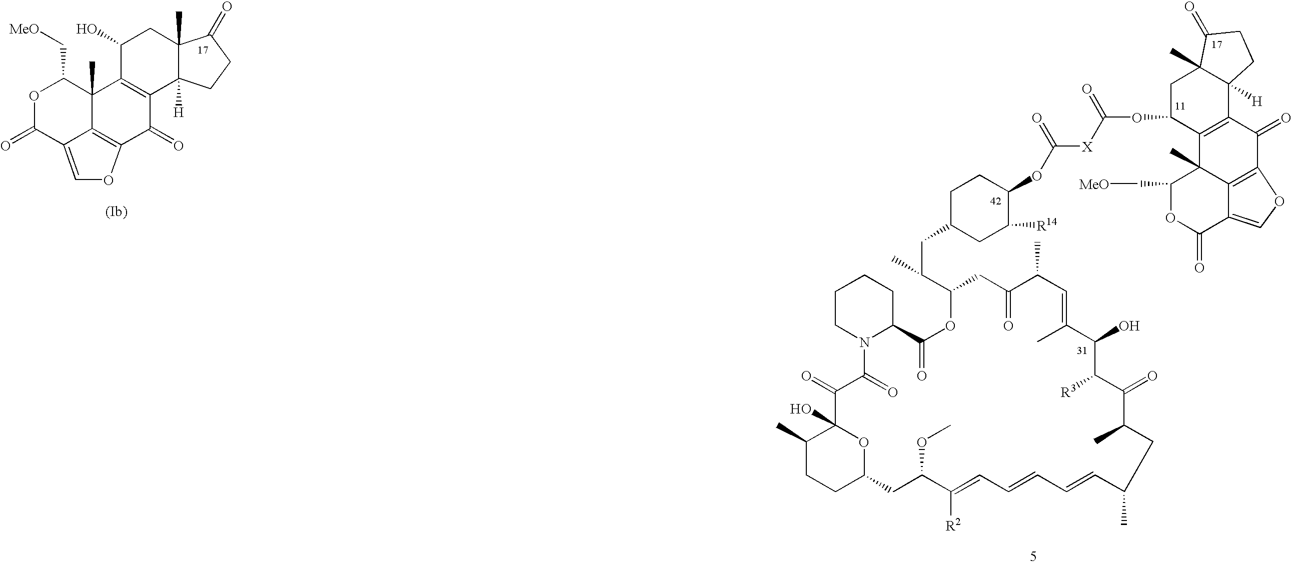

wherein R², R³, R¹⁴, X is as defined herein.

An exemplary conjugate which can be readily prepared by employing procedures described above include, but is not limited to the structure:

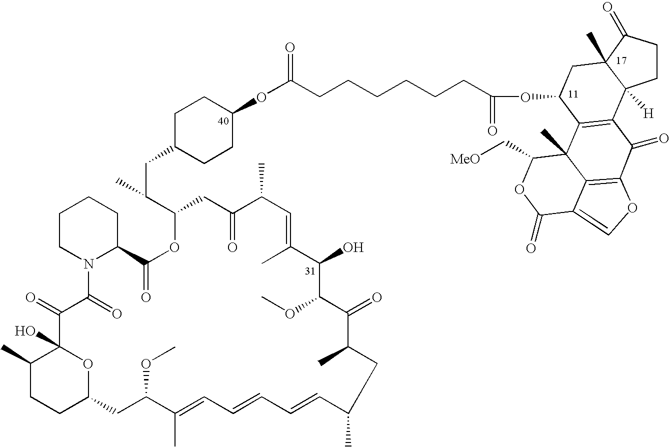

### 42,11′-linked wortmannin-suberate-41-desmethylrapalog conjugate

In one embodiment, 42,11′-linked wortmannin-rapalog conjugate 5 can be treated with R²¹ containing nucleophiles to give furan ring opened conjugate 6 as depicted in scheme 7.

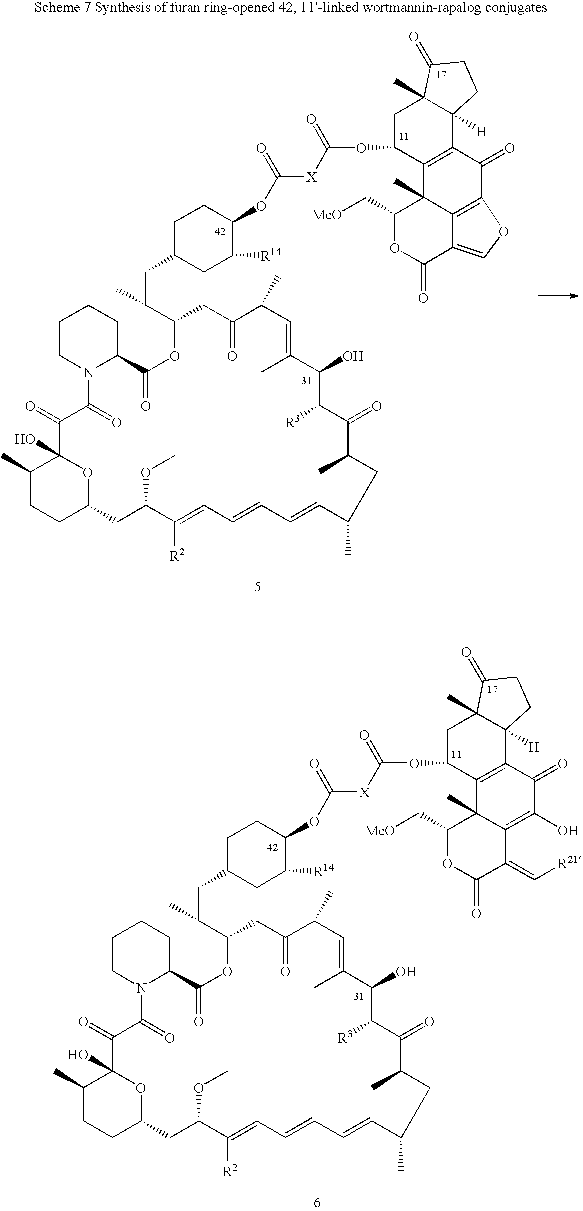

wherein, R², R³, R¹⁴, X and R^{21′} are as defined herein.

In one embodiment, the following exemplary compounds of formula may be prepared:

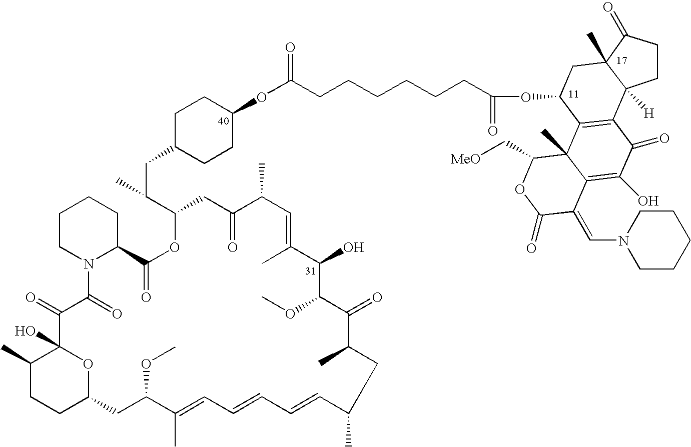

### 42,11-linked wortmannin-suberate-41-desmethoxyrapamycin conjugate, piperidine adduct

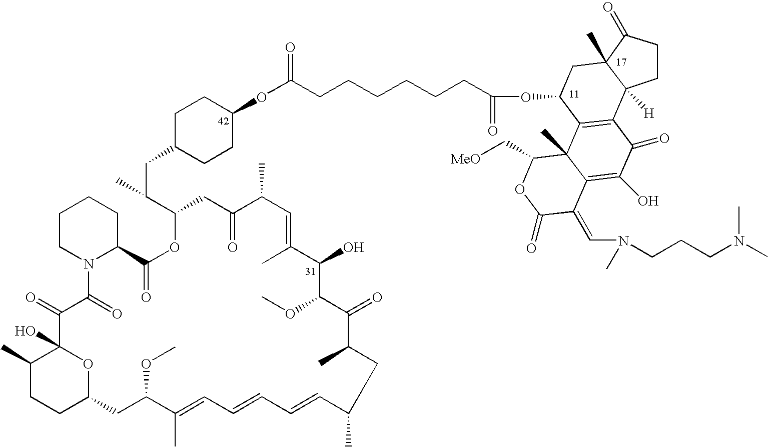

### 42,11′-linked wortmannin-suberate-41-desmethoxyrapamycin conjugate, N,N,N′-trimethyl 1,3-propanediamine adduct

In yet still another embodiment, the conjugates can be prepared according to the Scheme 8 through the linking position of rapalog 31-OH and wortmannin 11-OH. As for di-ester linked wortmannin-rapamycin, conjugates (7) are readily available by coupling 11-desacetyl wortmannin 11-dicarboxylic monoacid (Id) with rapalog 42-TBS ether (IIIg) in the presence of a coupling reagent such as, e.g., DCC, DIPC or EDC and a base such as DMAP, followed by de-protection (e.g., with diluted H₂SO₄) in excellent overall yield.

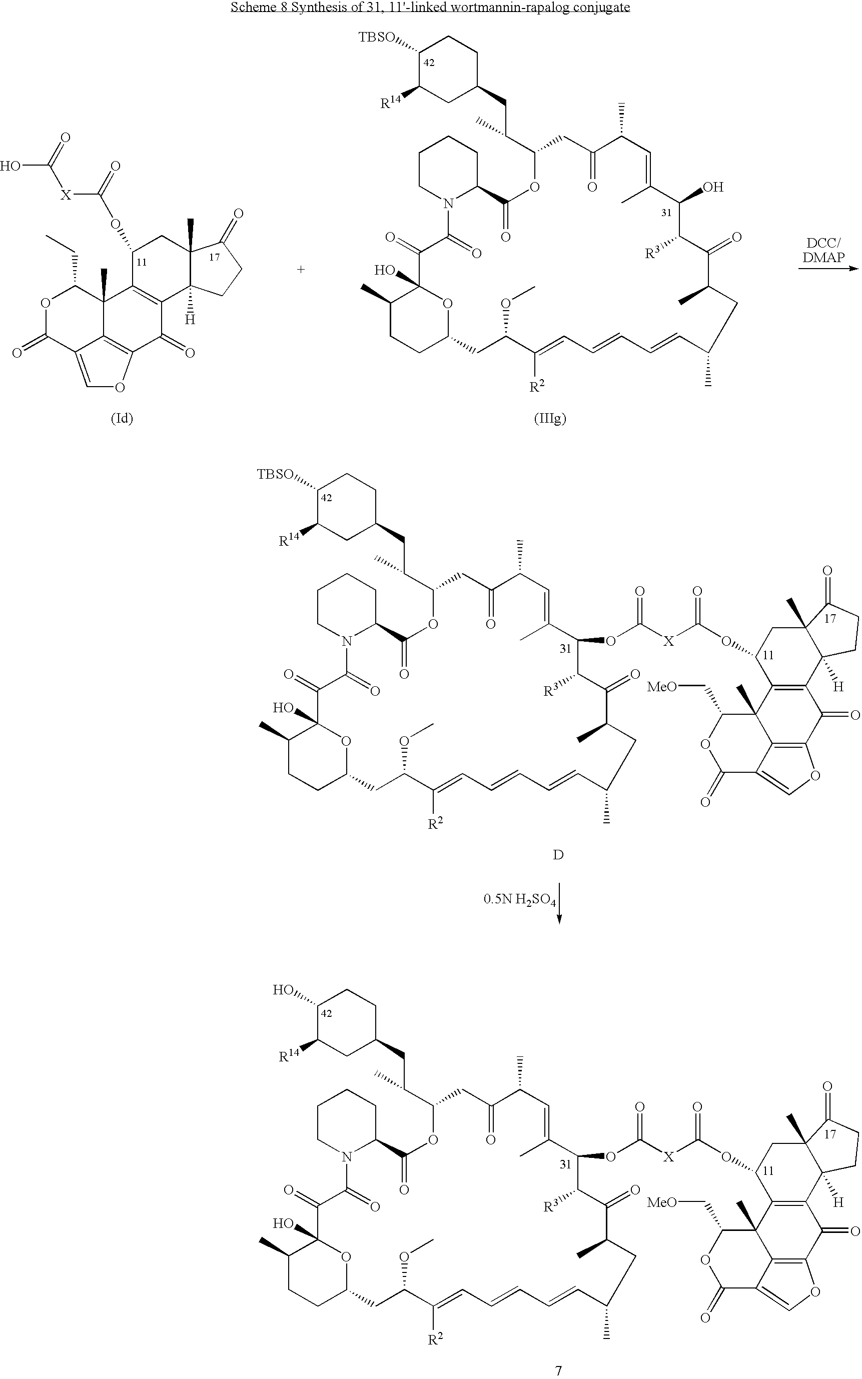

wherein, R², R³, R¹⁴, and X are as defined herein.

An exemplary conjugate which can be readily prepared by employing procedures described above includes:

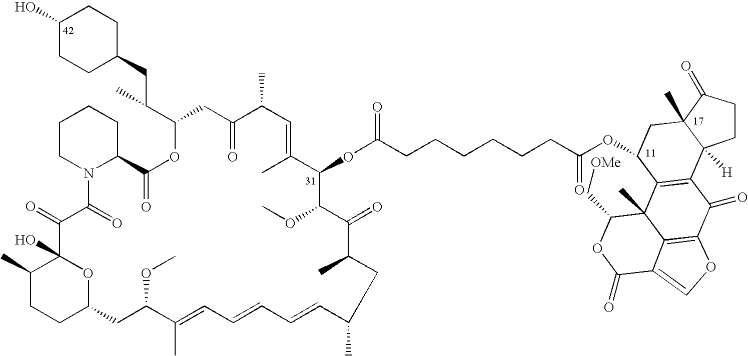

### 31,11′-linked wortmannin-suberate-41-desmethoxyrapamycin conjugate

In one embodiment, 31,11′-linked wortmannin-rapalog conjugate 7 can be treated with nucleophiles to give furan ring opened conjugate 8 as depicted in scheme 9.

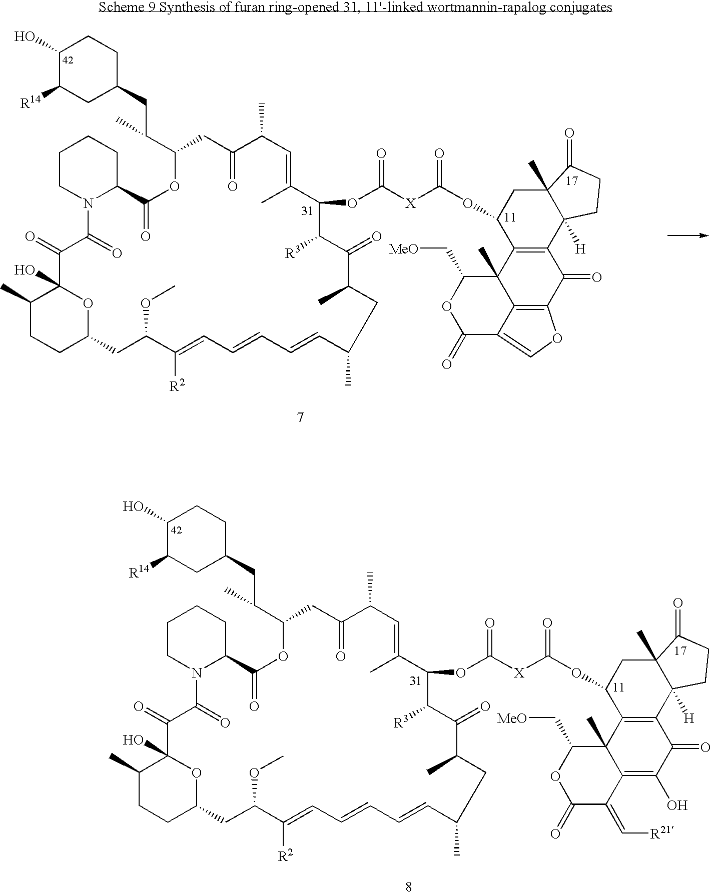

wherein, R², R³, R¹⁴, X and R^{21′} are as defined herein.

In one embodiment, the following exemplary compounds may be prepared:

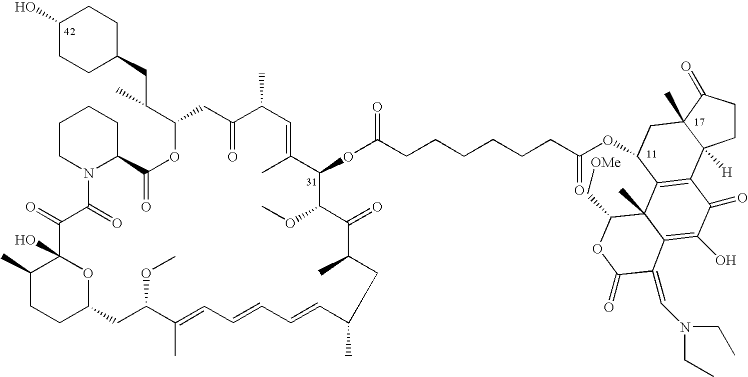

### 31,11′-linked wortmannin-suberate-41-desmethoxyrapamycin conjugate, diethylamine adduct

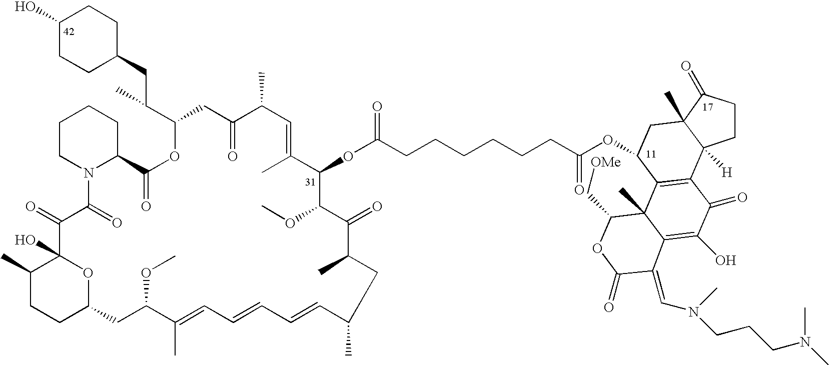

### 31,11-linked wortmannin-suberate-41-desmethoxyrapamycin conjugate, N,N,N′-trimethyl-1,3-propanediamine adduct

The presence of certain substituents in the conjugates described herein may enable salts of the conjugates to be formed. Suitable salts include pharmaceutically or physiologically acceptable salts, for example acid addition salts derived from organic or inorganic acids, and salts derived from inorganic or organic bases. Acid addition salt including, e.g., acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids. Salts derived from inorganic and organic bases include alkali metal salts such as sodium, lithium, or potassium, magnesium, calcium and organic amine salts such as dimethylamine, diethylamine, morpholine, and piperidine salts.

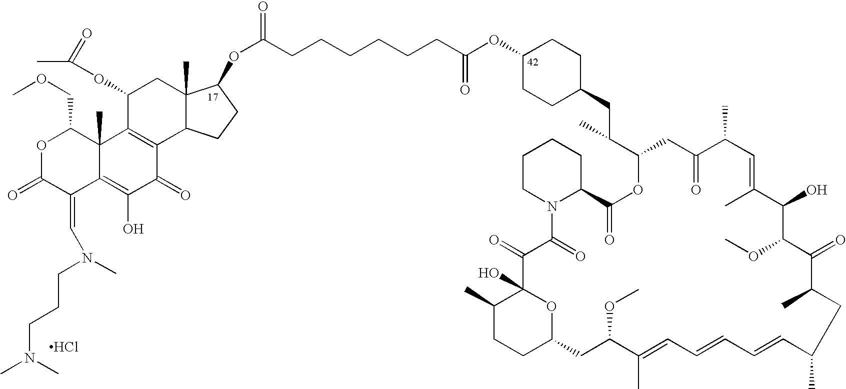

### 42,17′-linked wortmannin-suberate-41-desmethoxyrapamycin conjugate, N,N,N′-trimethyl propyldiamine adduct HCl salt

Salts and adducts can be readily selected by one of skill in the art. The conjugates, as well as the rapalog and wortmannin compounds may encompass tautomeric forms of the structures provided herein characterized by the bioactivity of the drawn structures.

The conjugates discussed herein also encompass “metabolites” which are unique products formed by processing the compounds by the cell or subject. Desirably, metabolites are formed in vivo.

In one embodiment, a salt and/or adduct of a free base conjugates described herein is desirable for improving the solubility, and thus, facilitating formulation of a conjugate. In another embodiment, improvement in the solubility is observed upon combination of a conjugate (e.g., a free base) with a buffer solution useful as a carrier for the conjugate. Such buffering solutions are described herein.

### Compositions and Uses of the Conjugates

In another aspect, the invention provides for the use of rapalog—L-wortmannin conjugates in preparing a pharmaceutical composition. Typically, such a composition contains, at a minimum, the conjugate and a pharmaceutically acceptable carrier.

In one embodiment, a conjugate is mixed with a physiologically compatible liquid carrier for delivery through a desired route. The carrier can be a solvent or dispersion medium containing, for example, water, ethanol, polyol (e.g., glycerol, propylene glycol and liquid polyethylene glycol), suitable mixtures thereof, and vegetable oils. In one embodiment, the carrier may be readily selected from among buffered saline solution (e.g., phosphate buffered saline, Hepes buffered saline, Tris-buffered saline), many of which are commercially available.

The pharmaceutical compositions may contain one or more excipients. Excipients are added to the composition for a variety of purposes.

Diluents increase the bulk of a solid pharmaceutical composition, and may make a pharmaceutical dosage form containing the composition easier for the patient and caregiver to handle. Diluents for solid compositions include, for example, microcrystalline cellulose (e.g. Avicel® reagent), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit® reagent), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel® reagent), hydroxypropyl methyl cellulose (e.g. Methocel® reagent), liquid glucose, magnesium aluminum silicate, maltodextrin, methylcellulose, polymethacrylates, povidone (e.g. Kollidon® and Plasdone® reagents), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol® and Primellose® reagents), colloidal silicon dioxide, croscarmellose sodium, crospovidone (e.g. Kollidon® and Polyplasdone® reagents), guar gum, magnesium aluminum silicate, methyl cellulose, microcrystalline cellulose, polacrilin potassium, powdered cellulose, pregelatinized starch, sodium alginate, sodium starch glycolate (e.g. Explotab® reagent) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Solid and liquid compositions may also be dyed using any pharmaceutically acceptable colorant to improve their appearance and/or facilitate patient identification of the product and unit dosage level.

In liquid pharmaceutical compositions, the conjugate and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

A liquid composition may also contain a buffer such as gluconic acid, lactic acid, citric acid or acetic acid, sodium gluconate, sodium lactate, sodium citrate or sodium acetate. Selection of excipients and the amounts used may be readily determined by the formulation scientist based upon experience and consideration of standard procedures and reference works in the field.

The solid compositions include powders, granulates, aggregates and compacted compositions. The dosages include dosages suitable for oral, buccal, rectal, parenteral (including subcutaneous, intramuscular, and intravenous), inhalant and ophthalmic administration. The most suitable administration in any given case will depend on the nature and severity of the condition being treated. The dosages may be conveniently presented in unit dosage form and prepared by any of the methods well-known in the pharmaceutical arts.

Dosage forms include solid dosage forms such as tablets, powders, capsules, suppositories, sachets, troches and lozenges, as well as liquid syrups, suspensions and elixirs.

The dosage form may be a capsule containing the composition, for example, a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling may include any of the aforementioned blends and granulates that were described with reference to tableting, however, they are not subjected to a final tableting step.

### Uses and Products

In another aspect, the invention provides an anti-neoplastic method comprising administering to a subject a pharmaceutically effective amount of a conjugate as described herein. Such a neoplasm is typically selected from among prostate cancer, breast cancer, renal cancer, colon cancer, ovarian cancer, glioma, soft tissue sarcoma, neuroendocrine tumor of the lung, cervical cancer, uterine cancer, head and neck cancer, glioblastoma, non-small cell lung cancer, pancreatic cancer, lymphoma, melanoma, and small cell lung cancer.

In a combination therapy, the conjugate may be administered before, during, or after commencing therapy with another agent, as well as any combination thereof, i.e., before and during, before and after, during and after, or before, during and after commencing the other anti-cancer therapy.

When the anti-cancer therapy is radiation, the source of the radiation can be either external (external beam radiation therapy) or internal (brachytherapy) to the patient being treated. The dose of anti-cancer therapy administered to the patient depends on numerous factors, including, for example, the type of agent, the type and severity of the tumor being treated and the route of administration of the agent.

Optionally, the method provides for administering the conjugate in a combination regimen with another active component. Such active component may be readily selected by one of skill in the art from among, e.g., an immunomodulator (e.g., an immunostimulatant or an immunosuppressant), an antineoplastic agent, or other desired component. When used in such a regimen, the conjugate may be administered prior to, simultaneously with, or following administration of the other active component. Further, the conjugate and the other active components may be delivered by the same route, or by different routes, of administration.

In one embodiment, the conjugate is administered in a regimen with an immunomodulator (e.g., an interferon, an interleukin (e.g., IL-2), or Bacillus Calmette-Guerin (BCG)). Suitable interferons are readily selected from among those known to those of skill in the art including, e.g., an interferon α, an interferon β, or an interferon γ. In one embodiment, the interferon is an interferon α. One interferon α (IFN α) is available commercially as “Intron-A”.

In another embodiment, the conjugate is administered in a regimen with an anti-VEGF monoclonal antibody. One suitable anti-VEGF monoclonal antibody is available, e.g., as AVASTIN.

As is typical with oncology treatments, dosage regimens are closely monitored by the treating physician, based on numerous factors including the severity of the disease, response to the disease, any treatment related toxicities, age, and health of the patient. Dosage regimens are expected to vary according to the route of administration.

Administration of the compositions may be oral, intravenous (i.v.), respiratory (e.g., nasal or intrabronchial), infusion, parenteral (besides i.v., such as intralesional, intraperitoneal and subcutaneous injections), intraperitoneal, transdermal (including all administration across the surface of the body and the inner linings of bodily passages including epithelial and mucosal tissues), and vaginal (including intrauterine administration). Other routes of administration are also feasible, such as via liposome-mediated delivery; topical, nasal, sublingual, uretheral, intrathecal, ocular or optic delivery, implants, rectally, intranasally.

It is projected that initial i.v. infusion dosages of the conjugate will be from about 5 to about 175 mg, or about 5 to about 25 mg, when administered on a weekly dosage regimen. It is projected that an oral dosage of a conjugate would be in the range of 10 mg/week to 250 mg/week, about 20 mg/week to about 150 mg/week, about 25 mg/week to about 100 mg/week, or about 30 mg/week to about 75 mg/week. For rapalog, the projected oral dosage will be between 0.1 mg/day to 25 mg/day. Precise dosages will be determined by the administering physician based on experience with the individual subject to be treated.

In one embodiment, further included is a product or pharmaceutical pack containing one or more container(s) having one, one to four, or more unit(s) of a conjugate in unit dosage form and optionally, another active agent (e.g., an interferon or anti-VEGF monoclonal antibody). Such a product may contain other components, including, e.g., a diluent, carrier, syringe, and/or instructions for administration of the conjugate. Typically, pharmaceutical packs contain an anti-neoplastic dosage regimen for an individual mammal.

In another embodiment, pharmaceutical packs contain a course of anti-neoplastic treatment for one individual mammal comprising a container having a unit of a rapalog-wortmannin conjugate in unit dosage form, and optionally, a container with another active agent. In some embodiments, the compositions are in packs in a form ready for administration. In other embodiments, the compositions of the invention are in concentrated form in packs, optionally with the diluent required to make a final solution for administration. In still other embodiments, the product contains a compound useful in the invention in solid form and, optionally, a separate container with a suitable solvent or carrier for the compound useful herein.

In still other embodiments, the above packs/kits include other components, e.g., instructions for dilution, mixing and/or administration of the product, other containers, syringes, needles, etc. Other such pack/kit components will be readily apparent to one of skill in the art.

### EXAMPLES

The following examples further illustrate the invention, but should not be construed to limit the scope of the invention in any way. It is to be understood and expected that variations in the principles of the invention herein disclosed may be made by one skilled in the art and it is intended that such modifications are to be included within the scope of the present invention.

### Example 1

### Synthesis of 42,17′-linked Rapalog-Wortmannin Conjugates

### Exemplary Example 1a

### 42,17′-linked 41-Desmethoxyrapamycin-Suberate-Wortmannin conjugate

To a solution of 17-hydroxywortmannin (430 mg, 1 mmol) in CH₂Cl₂ (10 mL) was added suberate anhydride (234 mg, 1.5 mmol), followed by DMAP (153 mg, 1.25 mmol). The mixture was then stirred at room temperature overnight. The crude mixture was purified by silica gel column eluting with hexane/acetone to give wortmannin 17-hemisuberate (200 mg) as a white powder. MS (ESI) m/e 609 (M+Na).

A mixture of wortmannin 17-hemisuberate (920 mg, 1.57 mmol), 31-OTMS-41-Desmethoxyrapamycin (1.20 g, 1.26 mmol) and catalytic amount of DMAP (77 mg) in 1,2-dichloroethane (10 mL) was cooled to 0 to 5° C. and was treated with 1,3-Diisopropylcarbodiimide (318 mg, 2.52 mmol). The mixture was stirred at 0 to 5° C. for 4 hours, then warmed to room temperature and stirred for 12 hours. The mixture was loaded on the silica gel column and eluted with hexane/EtOAc. The fractions containing product were collected and concentrated under vacuo to dryness.

The above product was dissolved in MeCN (12 mL) and cooled with an ice bath, diluted H₂SO₄ (0.5 N, 9 mL) was added dropwise and the mixture was stirred at 0-5° C. until the reaction completed (about 3.5 hours). EtOAc was added and the organic layer was separated. The organic layer was washed with water, 5% NaHCO₃ and brine. After solvent was evaporated under vacuum, the crude residue was purified on a silica gel column to give the desired product (1.54 g) as a white foam. MS (ESI) (M+Na)+1475.

The following representative compounds can be synthesized by employing the noted dicarboxylic anhydrides. See, Table 1.

TABLE 1

| Dicarboxylic | |
|---|---|
| Anhydride | Conjugate |
| suberate anhydride | 42,17′-linked 41-Desmethoxyrapamycin-Suberate- |
| | wortmannin conjugate |
| adipate anhydride | 42,17′-linked 41-Desmethoxyrapamycin-adipate- |
| | wortmannin conjugate |
| diglycolic anhydride | 42,17′-linked 41-Desmethoxyrapamycin-diglycoate- |
| | wortmannin conjugate |
| succinic anhydride | 42,17′-linked 41-Desmethoxyrapamycin-succinate- |
| | wortmannin conjugate |
| glutaric anhydride | 42,17′-linked 41-Desmethoxyrapamycin-glutarate- |
| | wortmannin conjugate |

### Example 2

### Synthesis of Amine Adducts of 42,17′-linked Rapalog-Wortmannin Conjugates

### General Procedure:

To a 0° C. solution of the 42,17-linked rapalog-wortmannin conjugate from example 1 in TBME was added a solution of amine in TBME. The mixture was stirred until the reaction was completed as monitored by thin layer chromatography (TLC) or high performance liquid chromatography (HPLC). The solvent was removed in vacuo. The products were purified either by trituration with solvents or via chromatography on silica gel eluting with CH₂Cl₂-MeOH.

### Exemplary Example 2a

### Diallylamine Adduct of 42,17′-linked 41-Desmethoxyrapamycin-Suberate-Wortmannin Conjugate

Diallylamine (64 mg, 0.66 mmol) was added to an ice-cold solution of 41-Desmethoxyrapamycin-suberate-wortmannin conjugate (552 mg, 0.38 mmol) from example 1a in TBME (15 mL). The mixture was then stirred at 0° C. for 40 hours. The mixture was concentrated to about 5 mL in vacuo and was triturated with hexane (30 mL). The product was collected on a Buchner funnel as a yellow powder (540 mg), MS: (M⁻) 1548.

### Exemplary Example 2b

### N,N,N′-trimethyl-1,3-propanediamine adduct of 42,17′-linked 41-Desmethoxyrapamycin-Suberate-Wortmannin conjugate

A solution of 41-Desmethoxyrapamycin-suberate-wortmannin conjugate (552 mg, 0.38 mmol) from example 1a in TBME (15 mL) was cooled to −30° C. N,N,N′-trimethyl-1,3-propanediamine (51 mg, 0.44 mmol) in TBME (3 mL) was added dropwise over 10 minutes. After addition, the mixture was stirred at −30° C. for 1 hr, then warmed to −20° C. and stirred for another 1 hour. Hexane (20 mL) was introduced while maintaining the temperature at −15° C. to −20° C. The product was collected on a Buchner funnel as a yellow powder (550 mg), MS: (M⁺) 1570

The following representative compounds can be synthesized by employing the appropriate rapalog-wortmannin conjugate and amines. See, Table 2.

TABLE 2

| | Rapalog-wartmannin | |
|---|---|---|
| Amine | conjugate | Adduct |
| N,N,N′- | 42,17′-linked 41- | N,N,N′-trimethyl-1,3- |
| trimethyl-1,3- | Desmethoxyrapamycin- | propanediamine adduct of |
| propanediamine | suberate-wortmannin | 42,17′-linked 41- |
| | | Desmethoxy rapamycin- |
| | | suberate-wortmannin |
| | | conjugate |
| diethylamine | 42,17′-linked 41- | diethylamine adduct of 42, |
| | Desmethoxyrapamycin- | 17′-linked 41- |
| | adipate-wortmannin | Desmethoxyrapamycin- |
| | | adipate-wortrmannin |
| | | conjugate |
| diallylamine | 42,17′-linked 41- | diallylamine adduct of 42, |
| | Desmethoxyrapamycin- | 17′-linked 41- |
| | adipate-wortmannin | Desmethoxyrapamycin- |
| | | adipate-wortmannin |
| | | conjugate |
| diallylamine | 42,17′-linked 41- | diallylamine adduct of 42, |
| | Desmethoxyrapamycin- | 17′-linked 41- |
| | glutarate-wortmannin | Desmethoxyrapamycin- |
| | | glutarate-wortmannin |
| | | conjugate |
| N,N,N′- | 42,17′-linked 41- | N,N,N′-trimethyl-1,3- |
| trimethyl-1,3- | Desmethoxyrapamycin- | propanediamine adduct of |
| propanediamine | succinate-wortmannin | 42,17′-linked rapamycin- |
| | | succinate-wortmannin |
| | | conjugate |
| N,N,N′- | 42,17′-linked 41- | N,N,N′-trimethyl-1,3- |
| trimethyl-1,3- | Desmethoxyrapamycin- | propanediamine adduct of |
| propanediamine | adipate-wortmannin | 42,17′-linked rapamycin- |
| | | adipate-wortmannin |
| | | conjugate |
| N,N,N′- | 42,17′-linked 41- | N,N,N′-trimethyl-1,3- |
| trimethyl-1,3- | Desmethoxyrapamycin- | propanediamine adduct of |
| propanediamine | glutarate-wortmannin | 42,17′-linked rapamycin- |
| | | glutarate-wortmannin |
| | | conjugate |

### Example 3

### Synthesis of 31,17′-linked Rapalog-Wortmannin Conjugates

### Exemplary Example 3a

### 31,17′-linked 41-Desmethoxyrapamycin-Suberate-Wortmannin Conjugate

A mixture of wortmannin 17-hemisuberate (146.5 mg, 0.25 mmol), 41-Desmethoxyrapamycin 42-OTBS (200 mg, 0.2 mmol) and a catalytic amount of DMAP (12.2 mg, 0.1 mmol) in 1,2-dichloroethane (2 mL) was cooled to 0-5° C. and was treated with 1,3-Diisopropylcarbodiimide (50.4 mg, 0.4 mmol). The mixture was stirred at 0 to 5° C. for 16 hours. Purification via silica gel to give 298 mg (95% yield) white powder.

This white powder (295 mg) was dissolved in MeCN (5 mL) and cooled with an ice-bath. 2N H₂SO₄ (2 mL) was added dropwise. After addition, the mixture was stirred for 2.5 hours at 0 to 5° C. EtOAc was added and the organic layer was separated. The organic layer was washed with water, 5% NaHCO₃ and brine, followed by evaporation of the solvent under vacuum. The crude product was purified on a silica gel column to give the desired product (242 mg, 89%) as white foam. MS (ESI): (M+Na)⁺ 1475.

The following representative compounds can be synthesized by employing the appropriate wortmannin 17-hemiacid. See, Table 3.

TABLE 3

| wortmannin 17-hemiacid | Conjugate |
|---|---|
| wortmannin 17-hemisuberate | 31,17′-linked 41-Desmethoxyrapamycin- |
| | Suberate-Wortmannin conjugate |
| wortmannin 17-hemiadipate | 31,17′-linked 41-Desmethoxyrapamycin- |
| | adipate-wortmannin conjugate |
| wortmannin 17- | 31,17′-linked 41-Desmethoxyrapamycin- |
| hemidiglycoate | diglycoate-wortmannin conjugate |
| wortmannin 17-hemisuccinate | 31,17′-linked 41-Desmethoxyrapamycin- |
| | succinate-wortmannin conjugate |
| wortmannin 17-hemiglutarate | 31,17′-linked 41-Desmethoxyrapamycin- |
| | glutarate-wortmannin conjugate |

### Example 4

### Synthesis of Amine Adducts of 31,17′-linked Rapalog-Wortmannin Conjugates

### General Procedure:

To a 0° C. solution of 31,17′-linked rapalog-wortmannin conjugate from example 3 in TBME was added a solution of amine in TBME. The mixture was stirred until reaction was completed as monitored by TLC or HPLC. The solvent was removed in vacuo. The products were purified either by trituration with solvents or via chromatography on silica gel eluting with CH₂Cl₂-MeOH.

### Exemplary Example 4a

### Diallylamine Adduct of 31,17′-linked 41-Desmethoxyrapamycin-Suberate-Wortmannin Conjugate

Diallylamine (3 mg) in TBME (0.1 mL) was added to an ice-cold solution of 31,17′-linked 41-Desmethoxyrapamycin-suberate-wortmannin conjugate (30 mg, 0.02 mmol) from example 3a in TBME (0.2 mL). The mixture was then stirred at 0° C. for 24 hours. The solvent was removed by N₂ flow and the residue was triturated with hexane (1 mL). The product was collected on a Buchner funnel as a yellow powder (30 mg), MS (ESI): (M+Na) 1572.

### Exemplary Example 4b

### N,N,N′-trimethyl-1,3-propanediamine Adduct of 31,17′-linked 41-Desmethoxyrapamycin-Suberate-Wortmannin Conjugate

A solution of 41-Desmethoxyrapamycin-suberate-wortmannin conjugate (40 mg, 0.028 mmol) from example 3a in TBME (0.2 mL)/CH₂Cl₂ (0.025 mL) was cooled to −30° C. N,N,N′-trimethyl-1,3-propanediamine (4 mg, 0.034 mmol) in TBME (0.1 mL) was added dropwise over 10 minutes. After addition, the mixture was stirred at −30° C. for 1 hour. Hexane (1 mL) was introduced while maintaining the temperature at −15° C. to −20° C. The product was collected on a Buchner funnel as a yellow powder (39 mg), MS: (M⁺) 1570.

### Example 5

### Synthesis of 42,11′-linked Rapalog-Wortmannin Conjugates

### Exemplary Example 5a

### Synthesis of 42,11′-linked 41-Desmethoxyrapamycin-suberate-wortmannin Conjugate

A mixture of wortmannin 11-hemisuberate (87 mg, 0.16 mmol), 41-Desmethoxyrapamycin 31-OTMS (134 mg, 0.14 mmol) and a catalytic amount of DMAP (8.5 mg, 0.07 mmol) in 1,2-dichloroethane (1.5 mL) was cooled to 0-5° C. and treated with 1,3-Diisopropylcarbodiimide (35.3 mg, 0.28 mmol). The mixture was stirred at 0-5° C. for 5 hours, then warmed to room temperature (RT) and stirred for another 12 hours. Purification via silica gel to give 140 mg (68% yield) white powder.

Above white powder (140 mg) was dissolved in MeCN (2 mL), cooled with an ice-bath, and 0.5N H₂SO₄ (1.5 mL) was added dropwise. After addition, the mixture was stirred for 3 hours at 0-5° C. EtOAc was added and the organic layer was separated. The organic layer was washed with water, 5% NaHCO₃ and brine, followed by evaporation of the solvent under vacuum. The crude product was purified on a silica gel column to give desired product as white foam (101 mg). MS (ESI): (M+Na)⁺ 1431.

The following representative compounds can be synthesized by employing the appropriate wortmannin 11-hemiacid. See, Table 4.

TABLE 4

| wortmannin 11-hemiacid | Conjugate |
|---|---|
| wortmannin 11-hemisuberate | 42,11′-linked 41-Desmethoxyrapamycin- |
| | suberate-wortmannin conjugate |
| wortmannin 11-hemiadipate | 42,11′-linked 41-Desmethoxyrapamycin- |
| | adipate-wortmannin conjugate |
| wortmannin 11- | 42,11′-linked 41-Desmethoxyrapamycin- |
| hemidiglycoate | diglycoate-wortmannin conjugate |
| wortmannin 11-hemisuccinate | 42,11′-linked 41-Desmethoxyrapamycin- |
| | succinate-wortmannin conjugate |
| wortmannin 11-hemiglutarate | 42,11′-linked 41-Desmethoxyrapamycin- |
| | glutarate-wortmannin conjugate |

### Example 6

### Synthesis of Amine Adducts of 42,11′-linked Rapalog-Wortmannin Conjugates

### General Procedure:

To a 0° C. solution of 42,11′-linked rapalog-wortmannin conjugate from example 5 in TBME was added a solution of amine in TBME. The mixture was stirred until reaction was completed as monitored by TLC or HPLC. The solvent was removed in vacuo. The products were purified either by trituration with solvents or via chromatography on silica gel eluting with CH₂Cl₂-MeOH.

### Exemplary Example 6a

### Piperidine Adduct of 42,11′-linked 41-Desmethoxyrapamycin-Suberate-Wortmannin Conjugate

A solution of 42,11′-linked 41-Desmethoxyrapamycin-suberate-wortmannin conjugate from example 6 (30 mg) in CH₂Cl₂ (0.2 mL) was cooled with an ice-bath and treated with piperidine (4 mg). The mixture was stirred for 30 minutes. The solvent was removed by a N₂ stream and the residue triturated with hexane (1 mL). The product was collected on a Buchner funnel as a yellow powder (30 mg). MS (ESI): (M⁻) 1492.

### Exemplary Example 6b

### N,N,N′-trimethyl-1,3-propanediamine Adduct of 42,11′-linked 41-Desmethoxyrapamycin-suberate-wortmannin

A solution of 42,11′-linked 41-Desmethoxyrapamycin-suberate-wortmannin conjugate from example 6 (30 mg) in TBME (0.2 mL) was cooled to −30° C. and treated with a solution of N,N,N′-trimethyl-1,3-propanediamine (4 mg) in TBME (0.1 mL). The mixture was stirred for 1 hour at −30° C. Hexane (1 mL) was added. After stirring for 10 minutes, the product was collected on a Buchner funnel as a yellow powder (31 mg). MS (ESI): (M⁺) 1525.

### Example 7

### Synthesis of 31,11′-linked Rapalog-Wortmannin Conjugates

### Exemplary Example 7a

### 31,11′-linked 41-Desmethoxyrapamycin-suberate-wortmannin Conjugate

A mixture of wortmannin 11-hemisuberate (87 mg, 0.16 mmol), 41-Desmethoxyrapamycin 42-OTBS (140 mg, 0.14 mmol) and a catalytic amount of DMAP (8.5 mg, 0.07 mmol) in 1,2-dichloroethane (1.5 mL) was cooled to 0-5° C. and treated with 1,3-Diisopropylcarbodiimide (35.3 mg, 0.28 mmol). The mixture was stirred at 0-5° C. for 4 hours, then warmed to RT and stirred for another 12 hours. Purification via silica gel afforded 202 mg (95% yield) of a white powder.

This white powder (200 mg) was dissolved in MeCN (3.6 mL) and cooled with an ice-bath. 2N H₂SO₄ (1.5 mL) was added dropwise. After addition, the mixture was stirred for 2 hours at 0 to 5° C. EtOAc was added and the organic layer was separated. The organic layer was washed with water, 5% NaHCO₃ and brine, followed by evaporation of the solvent under vacuum. The crude product was purified on a silica gel column to give desired product as white foam (142 mg, 77%). MS (ESI): (M+Na)⁺ 1431.

The following representative compounds were synthesized by employing the appropriate wortmannin 11-hemiacid. See, Table 5.

TABLE 5

| wortmannin 11-hemiacid | Conjugate |
|---|---|
| wortmannin 11-hemisuberate | 31,11′-linked 41-Desmethoxyrapamycin- |
| | suberate-wortmannin conjugate |
| wortmannin 11-hemiadipate | 31,11′-linked 41-Desmethoxyrapamycin- |
| | adipate-wortmannin conjugate |
| wortmannin 11- | 31,11′-linked 41-Desmethoxyrapamycin- |
| hemidiglycoate | diglycoate-wortmannin conjugate |
| wortmannin 11-hemisuccinate | 31,11′-linked 41-Desmethoxyrapamycin- |
| | succinate-wortmannin conjugate |
| wortmannin 11-hemiglutarate | 31,11′-linked 41-Desmethoxyrapamycin- |
| | glutarate-wortmannin conjugate |

### Example 8

### Synthesis of Amine Adducts of 31,11′-linked Rapalog-Wortmannin Conjugates

### General Procedure:

To a 0° C. solution of 31,11′-linked rapalog-wortmannin conjugate from example 7 in TBME was added a solution of amine in TBME. The mixture was stirred until reaction was completed as monitored by TLC or HPLC. The solvent was removed in vacuo. The products were purified either by trituration with solvents or via chromatography on silica gel eluting with CH₂Cl₂-MeOH.

### Exemplary Example 8a

### Diethylamine Adduct of 31,11′-linked 41-Desmethoxyrapamycin-Suberate-Wortmannin Conjugate

A solution of 31,11′-linked 41-Desmethoxyrapamycin-suberate-wortmannin conjugate from example 7a (30 mg) in CH₂Cl₂ (0.2 mL) was cooled with an ice-bath and treated with a solution diethylamine (2.2 mg) in CH₂Cl₂ (0.05 mL). The mixture was stirred for 1 hour. The solvent was removed by a N₂ stream and the residue was triturated with hexane (1 mL). The product was collected on a Buchner funnel as a yellow powder (28 mg). MS (ESI): (M⁺+Na) 1504.

### Exemplary Example 8b

### N,N,N′-trimethyl 1,3-propanediamine Adduct of 31,11′-linked 41-Desmethoxyrapamycin-Suberate-Wortmannin Conjugate

A solution of 31,11′-linked 41-Desmethoxyrapamycin-suberate-wortmannin conjugate from example 7a (40 mg) in TBME (0.2 mL) was cooled to −30° C. and treated with a solution of N,N,N′-trimethyl-1,3-propanediamine (4.5 mg) in TBME (0.1 mL). The mixture was stirred for 1 hour at −30° C. Hexane (1 mL) was added. After stirring for 10 minutes, the product was collected on a Buchner funnel as a yellow powder (39 mg). MS (ESI): (M⁺) 1525.

### Example 9

### Xenograft Tumor Efficacy Study Methods

This example illustrates the ability of the conjugates described herein over compounds known in the art to reduce tumor size.

Female nude mice at 10 weeks of age were inoculated subcutaneously on the flank with 200 μL U87MG (human glioblastoma) tumor cell suspension. U87MG cells were suspended in full growth media and were implanted at 10 million cells per mouse. Mice were staged when tumors reached approximately 200 mm³ in sizes. Tumor bearing mice at staging (day 0) were randomized into treatment groups (n=10). The conjugates described herein were formulated in D5W vehicle (glucose-water) and were dosed IV on day 0 and day 7. See, Table 6.

TABLE 6

| Conjugate | Example | Dosage |
|---|---|---|
| N,N,N′-trimethyl-1,3-propanediamine adduct | 2b | 15 mg/kg |
| of 42,17′-linked 41-Desmethoxyrapamycin- | | |
| suberate-wortmannin conjugate | | |
| 41-Desmethoxyrapamycin | reference | 10 mg/kg |
| | compound 1 | |
| diallylamine adduct of 17-hydroxywortmannin | reference | 5 mg/kg |
| | compound 2 | |
| a mixture of 41-Desmethoxyrapamycin/ | mixture of | 10 mg/kg/ |
| diallylamine adduct of 17-hydroxywortmannin | reference | 5 mg/kg |
| | compounds | |
| | 1 and 2 | |
| diallylamine adduct of 42,17′-linked 41- | 2a | 15 mg/kg |
| Desmethoxyrapamycin-suberate-wortmannin | | |
| conjugate | | |

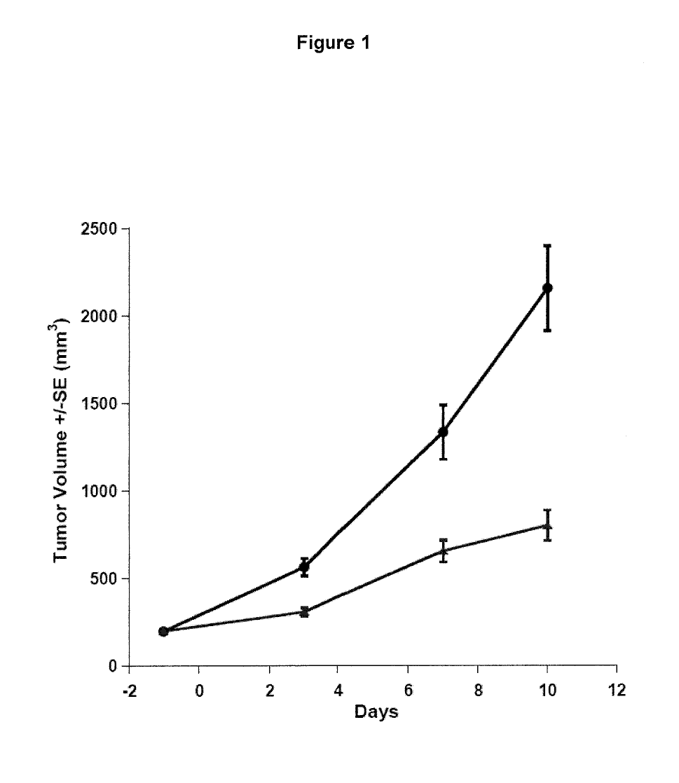

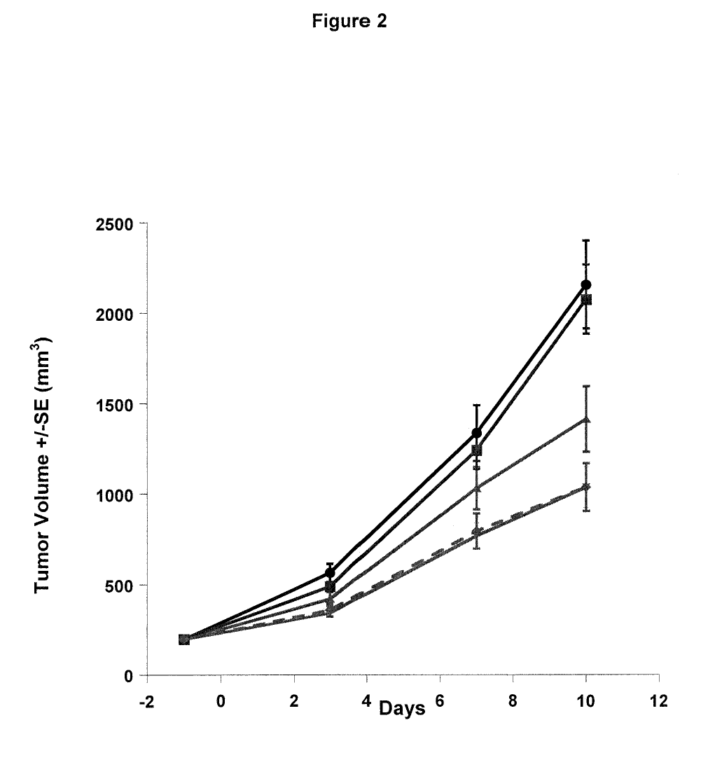

The growth of tumors was monitored twice a week for the duration of the experiment. Tumor size was measured using sliding vernier calipers, and the tumor mass was calculated using the formula (length×Width²)/2. Representative data is shown in FIGS. 1 and 2.

FIG. 1 provides a line graph illustrating the in vivo efficacy of N,N,N′-trimethyl-1,3-propanediamine adduct of 42,17′-linked 41-Desmethoxyrapamycin-suberate-wortmannin conjugate (conjugate of Example 2b; ▴). D5W vehicle (glucose-water) served as the control (). FIG. 2 provides a line graph illustrating the in vivo efficacy of the diallylamine adduct of 42,17-linked 41-Desmethoxyrapamycin-suberate-wortmannin conjugate (conjugate of Example 2a; (▾)) as compared with 41-Desmethoxyrapamycin (▴), diallylamine adduct of 17-hydroxywortmannin (▪), and a mixture (♦) of 41-Desmethoxyrapamycin/diallyl adduct of 17-hydroxywortmannin. D5W vehicle (glucose-water) served as the control ().

The compounds of Examples 2a and 2b resulted in smaller tumors than tumors treated with control or with reference compound 1 or reference compound. Further, the compounds of Examples 2 and 2b resulted in tumors of comparable size to that achieved with a mixture of reference compounds 1 and 2.

All publications cited in this specification are incorporated herein by reference. While the invention has been described with reference to particular embodiments, it will be appreciated that modifications can be made without departing from the spirit of the invention. Such modifications are intended to fall within the scope of the appended claims.

1. A conjugate having the formula or a pharmaceutically acceptable salt thereof: 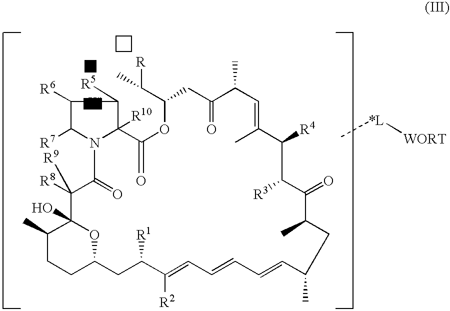
wherein:
L is a linker which is bound to the core structure of the rapalog of formula (III);
WORT is a wortmannin;
R¹ is selected from the group consisting of OH, an ester, an ether, and a point of attachment to L;
R² is methyl or H;
R³ is selected from the group consisting of H, OH, an ester, an ether, and a point of attachment to L;
R⁴ is selected from the group consisting of OH, an ester, an ether, an amide, a carbonate, a carbamate, and a phosphate;
R⁵, R⁶, and R⁷ are independently selected from the group consisting of H, alkyl, halogen, and hydroxyl;
R⁸,R⁹ is H, H or ═O;
R¹⁰ is selected from the group consisting of H, alkyl, halogen and hydroxyl;
X″ is a bond or CHR¹¹; or
CHR⁵—X″—CHR⁶— is: 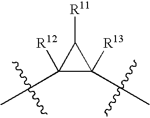
R¹¹, R¹², and R¹³ are independently selected from the group consisting of H, alkyl, halogen, and hydroxyl;
R is selected from the group consisting of formula A, formula B, formula C, formula D, formula E, formula F and point of attachment to L: 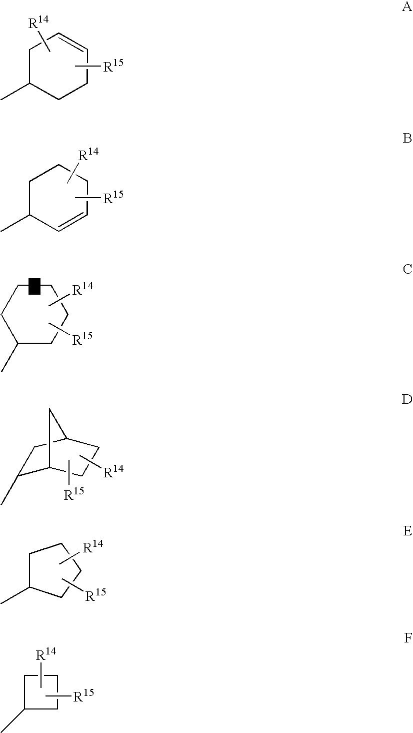
R¹⁴ and R¹⁵ are independently selected from the group consisting of H, OH, halogen, thiol, amine, alkyl, an ester, an ether, an amide, a carbonate, a carbamate, a sulfonate, a phosphate, a tetrazole, and point of attachment to L;
Y is a bond or CHR¹⁶;
R¹⁶ is selected from the group consisting of H, alkyl, halogen, hydroxyl, and a point of attachment to L;
wherein at least one of R, R¹, R³, R⁴, R¹⁴, R¹⁵ and R¹⁶ is a point of attachment of L to formula (III).

2. The conjugate according to claim 1, providing that formula (III) does not contain the combination of substitutents selected from the group consisting of:
(a) R¹ is OCH₃, R² is CH₃, R³ is OCH₃, R⁴ is OH, R⁵ is H, R⁶ is H, R⁷ is H, R⁸ and R⁹ are keto, R¹⁰ is H, X is CH₂, R is formula C, Y is a bond, R¹⁴ is cis-3-OMe, and R¹⁵ is trans-4-OH;
(b) R¹ is OCH₃, R² is CH₃, R³ is OCH₃, R⁴ is OH, R⁵ is H, R⁶ is H, R⁷ is H, R⁸ and R⁹ are keto, R¹⁰ is H, X is CH₂, R is formula C, Y is a bond, R¹⁴ is cis-3-OMe, and R¹⁵ is a trans-4-ether;
(c) R¹ is OCH₃, R² is CH₃, R³ is OCH₃, R⁴ is OH, R⁵ is H, R⁶ is H, R⁷ is H, R⁸ and R⁹ are keto, R¹⁰ is H, X is CH₂, R is formula C, Y is a bond, R¹⁴ is cis-3-OMe, and R¹⁵ is a trans-4-ester;
(d) R¹ is OCH₃, R² is CH₃, R³ is OCH₃, R⁴ is OH, R⁵ is H, R⁶ is H, R⁷ is H, R⁸ and R⁹ are keto, R¹⁰ is H, X is CH₂, R is formula C, Y is a bond, R¹⁴ is cis-3-OMe, and R¹⁵ is cis-4-tetrazole;
(e) R¹ is OCH₃, R² is CH₃, R³ is OCH₃, R⁴ is OH, R⁵ is H, R⁶ is H, R⁷ is H, R⁸ and R⁹ are keto, R¹⁰ is H, X is CH₂, R is formula C, Y is a bond, R¹⁴ is cis-3-OMe, and R¹⁵ is trans-4-phosphate;
(f) R¹ is OCH₃, R² is CH₃, R³ is OCH₃, R⁴ is OH, R⁵ is H, R⁶ is H, R⁷ is H, R⁸ and R⁹ are keto, R¹⁰ is H, X is CH₂, R is formula C, Y is a bond, R¹⁴ is cis-3-OH, and R¹⁵ is trans-4-OH;
(g) R¹ is OCH₃, R² is CH₃, R³ is OH, R⁴ is OH, R⁵ is H, R⁶ is H, R⁷ is H, R⁸ and R⁹ are keto, R¹⁰ is H, X is CH₂, R is formula C, Y is a bond, R¹⁴ is cis-3-OMe, and R¹⁵ is trans-4-OH;
(h) R¹ is OH, R² is CH₃, R³ is OCH₃, R⁴ is OH, R⁵ is H, R⁶ is H, R⁷ is H, R⁸ and R⁹ are keto, R¹⁰ is H, X is CH₂, R is formula C, Y is a bond, R¹⁴ is cis-3-OMe, and R¹⁵ is trans-4-OH; and
(i) R¹ is OCH₃, R² is CH₃, R³ is OCH₃, R⁴ is OH, R⁵ is H, R⁶ is H, R⁷ is H, R⁸ and R⁹ are keto, R¹⁰ is H, X is bond, R is formula C, Y is a bond, R¹⁴ is cis-3-OMe, and R¹⁵ is trans-4-OH.

3. The conjugate according to claim 2, wherein said trans-4-ether is trans-4-O—CH₂CH₂—OH and trans-4-ester is trans-4-O—COC(CH₃)(CH₂OH)₂.

4. The conjugate according to claim 1, wherein L is removed in whole or part in vivo from one or both of Rapalog or Wort.

5. The conjugate according to claim 1, wherein L is hydrolysable or enzymatically cleaved.

6. The conjugate according to claim 1, wherein L is characterized by formula (V):
-Z¹-X-Z²- (V)
wherein:
Z¹ and Z² are independently selected from the group consisting of a bond, —O—, N(R⁰), —S—, —OC(═O)—, —OC(═O)O—, —N(R⁰)C(═O), —OC(═O)N(R⁰)—, —N(R⁰)C(═O)N(R⁰)—, —OC(═S)N(R⁰)—, —N(R⁰)C(═S)N(R⁰)—; and ═N—N(R⁰)—;
R⁰ is at each occurrence independently selected from the group consisting of H, alkyl, alkenyl, and aryl; and
X is selected from the group consisting of cycloalkyl, aryl, alkylarylalkyl, heteroaryl, a heterocyclic group, a hydrocarbon chain having 1 to 16 carbon atoms which may be branched, unbranched, saturated or unsaturated, may be optionally substituted with one or more oxy, amine, sulfide, alkyl, alkenyl, aryl, alkoxy, hydroxyl, and halogen, and may be optionally interrupted by one or more ether (—O—), amine (—NH—), sulfide (—S—), —S(O)ₙ—, NR^{O}, —C(═O)N(R⁰)—, —OC(═O)N(R⁰)—, —N(R⁰)C(═O)N(R⁰)—, —OC(═S)N(R⁰)—, —N(R⁰)C(═S)N(R⁰)—, or ═N—N(R⁰), or a combination thereof.

7. The conjugate according to claim 6, wherein when Z¹ or Z² is selected from the group consisting of —O—, NR⁰, —S—, —OC(═O)—, —OC(═O)O—, —N(R⁰)C(═O)—, —OC(═O)N(R⁰)—, —N(R⁰)C(═O)N(R⁰)—, —OC(═S)N(R⁰)—, —N(R⁰)C(═S)N(R⁰)—, and ═N—N(R⁰)—, the group through which L is bound to the rapalog or WORT does not provide a further O group.

8. The conjugate according to claim 6, wherein X is selected from the group consisting of an alkyl chain of 1 to 16 carbon atoms interrupted by one or more groups selected from the group consisting of —O—, —S(O)ₙ—, NR⁰, —OC(═O)—, —OC(═O)O—, —C(═O)N(R⁰)—, and —OC(═OC)N(R⁰)—, and n is 0 to 2.

9. The conjugate according to claim 6, wherein L has the formula:

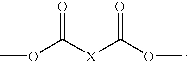

10. The conjugate according to claim 9, wherein:
X is a hydrocarbon chain of the formula —(CH₂)ₙ—, where n is 1 to 16; or
X is a hydrocarbon chain interrupted by an ether linkage of the formula:
—(CH₂)ₙ—O—(CH₂)ₙ—, where n is 1 to 8.

11. The conjugate according to claim 6, wherein Z¹ and Z² are a bond and X is an alkyl chain of 1 to 10 carbon atoms or an alkyl chain of 1 to 10 carbon atoms substituted with one, two, or more oxy groups.

12. The conjugate according to claim 6, wherein X is selected from the group consisting of C₁-C₈ alkyl, (CH₂CH₂O)ₙ, —(CH₂)ₙ—O—(CH₂)ₙ—, C₂-C₈ alkenyl, cycloalkyl, aryl, and a heterocyclic group and n is 1 to 8.

13. The conjugate according to claim 12, wherein X is selected from the group consisting of (CH₂)₂, (CH₂OCH₂), (CH₂)₃, (CH₂)₄, (CH₂)₅ and (CH₂)₆.

14. The conjugate according to claim 1, wherein L is bound to the rapalog formula (III) through one of R, R¹, R³, R⁴, R¹⁴ or R¹⁵.

15. The conjugate according to claim 1, wherein the rapalog is formula (IIIa), (IIIb), (IIIc), or (IIId):
(i) 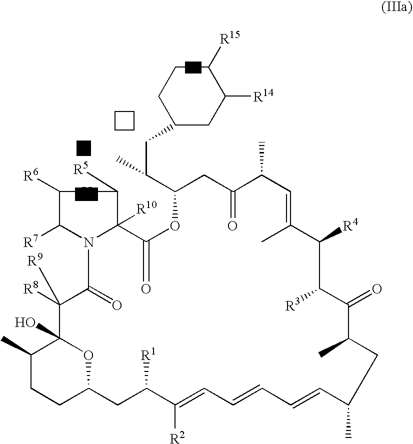
wherein Y is CHR¹⁶ or a bond;
(ii) 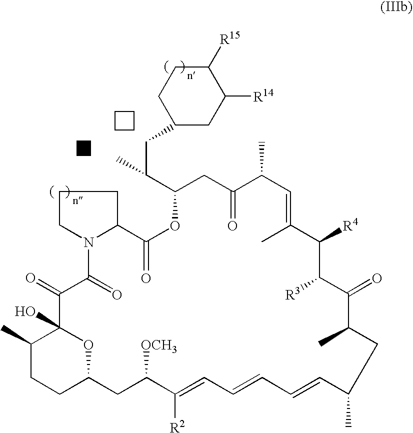
wherein n′ and n″ are independently 1 or 2.
(iii) 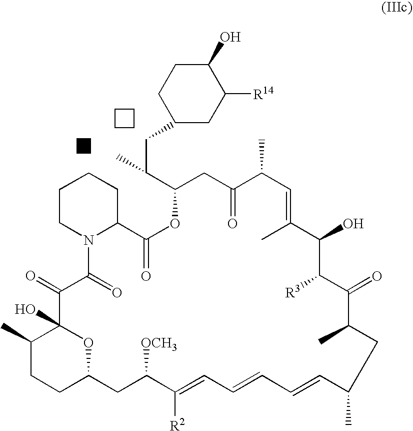
wherein R¹⁴ is H, halogen, thiol, amine, or alkyl; and
(iv)

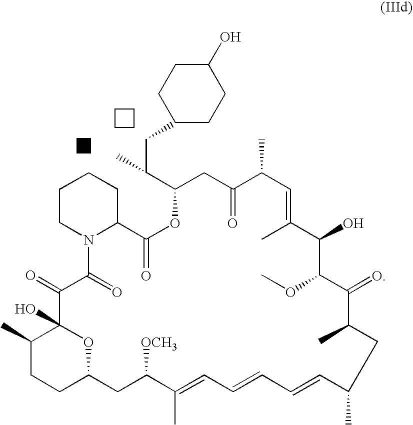

16. The conjugate according to claim 1, wherein the wortmannin has the core structure (Ia): 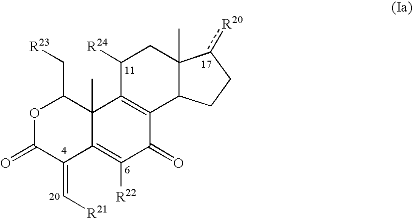
wherein:
R²⁰ is selected from the group consisting of O, OH, an ester, a carbonate, a carbamate, an ether, and a point of attachment to L;
R²¹ and R²² are bound together via an O heteroatom; or
R²¹ is selected from the group consisting of an ester, an ether, a thioether, a thioester, an amino, and a point of attachment to L;
R²² is selected from the group consisting of OH, an ester, a carbonate, a carbamate, an ether, and a point of attachment to L;
R²³ is selected from the group consisting of OH, an ester, an ether, and a point of attachment to L;
R²⁴ is selected from the group consisting of O, OH, an ester, a carbonate, a carbamate, and a point of attachment to L;
wherein at least one of R²⁰, R²¹, R²², R²³, and R²⁴ is the point of attachment to L.

17. The conjugate according to claim 16, wherein L is bound to the wortmannin core through one of R²⁰, R²¹, R²², R²³, or R²⁴.

18. The conjugate according to claim 16, wherein R²¹ has the formula NR^{a}R^{b},
wherein R^{a} and R^{b} are independently selected from the group consisting of H, alkyl, alkenyl, alkynyl, -(alkyl)-O-(alkyl)-, -(alkyl)-NR^{c}R^{d}, -(alkyl)-C(═O)NR^{c}R^{d}—, cycloalkyl, aryl, and a heterocyclic group; with the proviso that both R^{a} and R^{b} are not H;
or R^{a} and R^{b} may be taken together to form a three to seven membered heterocyclic ring having up to 3 heteroatoms which is optionally substituted by 1 to 3 substituents independently selected from the group consisting of halogen, hydroxyl, thio, alkyl, alkenyl, alkoxy, oxo, amino, cyano, C₁-C₃ perfluoroalkyl, alkylaryl, alkylheteroaryl, aryl, and heteroaryl;
R^{c} and R^{d} are independently selected from the group consisting H, alkyl, alkenyl, alkynyl, aryl, cycloalkyl, and heterocycyl; or
R^{c} and R^{d} are taken together to form a three to seven membered cyclic or heterocyclic ring having up to 3 heteroatoms which is optionally substituted by 1 to 3 substituents independently selected from the group consisting of halogen, hydroxyl, thio, alkyl, alkenyl, alkoxy, oxo, amino, cyano and C₁-C₃ perfluoroalkyl.

19. The conjugate according to claim 18, wherein:
R^{a} is H and R^{b} is phenyl; or
R^{a} and R^{b} are lower alkyl.

20. The conjugate according to claim 18, wherein R²¹ is selected from the group consisting of diethylamine, diallylamine, N,N,N′-trimethyl-1,3-propanediamine, piperidine, and N,N-dimethyl-N′-ethyl-ethylenediamine and R¹³ is —OH.

21. The conjugate according to claim 1, wherein R¹⁴ and R¹⁵ are independently selected from the group consisting of H, OH, halogen, thiol, amine, alkyl, an ester, an amide, a carbonate, a carbamate, a sulfonate, a phosphate, a tetrazole, and point of attachment to L.

22. The conjugate according to claim 1, wherein said conjugate is selected from the group consisting of:
42,17′-linked wortmannin-succinate-41-Desmethoxyrapamycin conjugate;
42,17′-linked wortmannin-glutarate-41-Desmethoxyrapamycin conjugate;
42,17′-linked wortmannin-adipate-41-Desmethoxyrapamcyin conjugate;
42,17′-linked wortmannin-suberate-41-Desmethoxyrapamycin conjugate;
42,17′-linked wortmannin-diglycolinate-41-Desmethoxyrapamycin conjugate;
42,17′-linked wortmannin-adipate-41-Desmethoxyrapamycin conjugate, diethylamine adduct;
42,17′-linked wortmannin-adipate-41-Desmethoxyrapamycin conjugate, diallylamine adduct;
42,17′-linked wortmannin-succinate-41-Desmethoxyrapamycin conjugate, N,N,N′-trimethyl 1,3-propanediamine adduct;
42,17′-linked wortmannin-adipate-41-Desmethoxyrapamycin conjugate, N,N,N′-trimethyl 1,3-propanediamine adduct;
42,17′-linked wortmannin-suberate-41-Desmethoxyrapamycin conjugate, N,N,N′-trimethyl 1,3-propanediamine adduct;
42,17′-linked wortmannin-suberate-41-Desmethoxyrapamycin conjugate, diallylamine adduct;
42,17′-linked wortmannin-suberate-41-Desmethoxyrapamycin conjugate, N,N,N′-trimethyl propyldiamine adduct HCl salt;
31,17′-linker wortmannin-suberate-41-Desmethoxyrapamycin conjugate;
31,17′-linked wortmannin-suberate-41-Desmethoxyrapamycin conjugate, N,N,N′-trimethyl 1,3-propanediamine adduct;
31,17′-linked wortmannin-suberate-41-Desmethoxyrapamycin conjugate, diallylamine adduct;
42,11′-linked wortmannin-suberate-41-Desmethoxyrapamycin conjugate;
42,11′-linked wortmannin-suberate-41-Desmethoxyrapamycin conjugate, piperidine adduct;
42,11′-linked wortmannin-suberate-41-Desmethoxyrapamycin conjugate, N,N,N′-trimethyl-1,3-propanediamine adduct;
31,11′-linked wortmannin-suberate-41-Desmethoxyrapamycin conjugate;
31,11′-linked wortmannin-suberate-41-Desmethoxyrapamycin conjugate, diethylamine adduct; and
31,11-linked wortmannin-suberate-41-Desmethoxyrapamycin conjugate, N,N,N′-trimethyl-1,3-propanediamine adduct.

23. A pharmaceutical composition comprising a conjugate of claim 1 and a pharmaceutically acceptable carrier.

24. A method of treating a neoplasm comprising administering to a subject a pharmaceutically effective amount of a conjugate of claim 1.

25. The method according to claim 24, wherein the neoplasm is selected from the group consisting of prostate cancer, breast cancer, renal cancer, colon cancer, ovarian cancer, glioma, soft tissue sarcoma, neuroendocrine tumor of the lung, cervical cancer, uterine cancer, head and neck cancer, glioblastoma, non-small cell lung cancer, pancreatic cancer, lymphoma, melanoma, and small cell lung cancer.

26. The method according to claim 24, wherein the conjugate is administered intravenously or directly to the neoplasm.
